(19) United States
(12) Patent Application Publication
(10) Pub. No.: US 2013/0230580 A1
(43) Pub. Date: Sep. 5, 2013

(54) Administration of SNS Neuroprotective Agents to Promote Hematopoietic Regeneration

(71) Applicants: Paul S. Frenette, New York, NY (US); Daniel Lucas-Alcaraz, New York, NY (US)

(72) Inventors: Paul S. Frenette, New York, NY (US); Daniel Lucas-Alcaraz, New York, NY (US)

(21) Appl. No.: 13/823,578

(22) PCT Filed: Sep. 14, 2011
(86) PCT No.: PCT/US11/51640

§ 371 Date: May 15, 2013

Related U.S. Application Data
(60) Provisional application No. 61/382,883, filed on Sep. 14, 2010.

(51) Int. Cl. A61K 31/05; A61K 38/18; A61K 39/395

(52) U.S. Cl. CPC A61K 31/05; A61K 39/395; A61K 38/185
USPC 424/450; 514/734; 424/134.1; 424/464; 424/451; 424/499; 424/491; 424/468; 424/457

(57) ABSTRACT

Provided are therapeutics, uses and methods in which neuro-regenerative therapy using neuroprotective agents, or anti-neuropathic agents, to prevent loss or restore hematopoietic capacity and progenitor mobilization.

This invention was made with government support under grant numbers R01 DK056638 and R01 HL69438 awarded by the National Institute of Health/National Institute of Diabetes and Digestive and Kidney Diseases (NIH/NIDDK) and under grant number 1F30HL099028-01 awarded by the National Heart, Lung, and Blood Institute (NIH/NHLBI). The government has certain rights in the invention.

### FIELD OF THE DISCLOSURE

The disclosure relates to the field of medical treatment of disorders in man and other animals. In particular, the disclosure relates to the maintenance and regeneration of hematopoietic capacity during and after administration of a cytotoxic agent.

### BACKGROUND

Anti-cancer chemotherapy drugs challenge hematopoietic tissues to regenerate, but commonly produce long-term sequelae. Deficits in hematopoietic stem or stromal cell function have been described, but the mechanisms mediating chemotherapy-induced hematopoietic dysfunction remain unclear. Administration of multiple cycles of cisplatin chemotherapy causes significant sensory neuropathy, compromises hematopoietic regeneration after stress, and reduces progenitor mobilization.

Tissue regeneration operates through diverse modes and mechanisms among animal phyla. In mammals, individual organs exhibit broad differences in regenerative potential. For example, regeneration appears very limited in the postnatal heart and brain but more vigorous in the liver and skin. The hematopoietic system continuously renews itself; billions of blood cells are produced every day in the bone marrow (BM) by the regulated proliferation and differentiation of hematopoietic stem cells (HSC). Fate decisions are orchestrated by specific interactions of HSC and committed progenitors with their microenvironment. Anti-cancer chemotherapy and preparative regimens for bone marrow transplantation present a robust regenerative challenge since these protocols often lead to profound bone marrow aplasia followed by extensive remodeling of the stromal compartment to recover normal hematopoiesis. In addition to the acute cytotoxicity, patients that have received prior chemotherapy often exhibit irreversible chronic BM damage leading to impaired hematopoietic reserve. Functional defects in HSC and/or stromal cell activities have been reported following conventional chemotherapy, but the mechanisms that cause permanent damage to HSC function remain unresolved.

Compromised HSC mobilization in patients that have received prior cytotoxic therapy has been well documented. Several chemotherapeutic drugs (e.g., vinca alkaloids, taxanes, platinum-based) commonly induce peripheral neuropathies that can limit dosage and, consequently, the effectiveness of the treatment.

For all of the foregoing reasons, needs continue to exist in the art for therapeutics and/or prophylactics effective in inhibiting or preventing a loss or reduction in hematopoietic capacity.

### SUMMARY

The disclosure provides a solution to at least one of the aforementioned problems in the art in providing methods for maintaining hematopoietic capacity and methods for promoting hematopoietic regeneration in subjects exposed to conditions that compromise hematopoiesis, such as cancer treatment by chemo- and/or radio-therapy, or treatment of various diseases, disorders or conditions with cytotoxins. The disclosure establishes that hematopoietic defects are caused by damage to adrenergic nerve fibers that innervate the bone marrow. Furthermore, neuro-regenerative therapy using 4-methylcatechol or glial-derived neurotrophic factor (GDNF) restored hematopoietic recovery and progenitor mobilization. Thus, adrenergic signals critically contribute to bone marrow regeneration. These data shed light on the potential benefit of neuroprotection to shield hematopoietic niches.

In one aspect, the disclosure provides a method of promoting hematopoietic regeneration in a subject comprising administering an effective amount of a sympathetic nervous system neuroprotective agent.

Another aspect provides a method of reducing a loss of hematopoietic regeneration capacity in a subject comprising administering an effective amount of a sympathetic nervous system neuroprotective agent.

In some embodiments of the method of promoting hematopoietic regeneration or the method of reducing a loss of hematopoietic regeneration capacity, the neuroprotective agent is selected from the group consisting of 4-methylcatechol (4-MC), Glial cell-Derived Neurotrophic Factor, Glial cell-Derived Neurotrophic Factor fusion protein, interleukin-6, insulin growth factor, neural growth factor, vitamin E, glutathione leukemia inhibitory factor, acetylcysteine, acetyl-L-carnitine, amifostine, glutathione, oxcarbazepine, E2072, 2-(phosphonomethyl) pentanedioic acid, 2-(3-mercaptopropyl)pentanedioic acid, Trypanosoma cruzi trans-sialidase/parasite-derived neurotrophic factor, Brain-Derived Neurotrophic Factor, Transforming Growth Factor-β, cardiotrophin-1, Insulin-like Growth Factor-1, basic Fibroblast Growth Factor, Vascular Endothelial Growth Factor, Hepatocyte Growth Factor Neurotrophin 3, Neurotrophin 4/5, platelet-rich plasma, pifithrin, Z-1-117, 2-imino-2,3,4,5,6,7-hexahydrobenzothiazole derivatives, 2-imino-2,3,4,5,6,7-hexahydrobenzoxazole derivatives, Gambogic amide, amitriptyline, 7,8-dihydroxyflavone, neurturin, artemin, and persephinm.

In some embodiments of either of the above methods, i.e., the method of promoting hematopoietic regeneration or the method of reducing a loss of hematopoietic regeneration capacity, the following feature or features are found. The neuroprotective agent is or may be selected from the group consisting of Glial Cell-Derived Neurotrophic Factor, a Glial Cell-Derived Neurotrophic Factor fusion protein, 4-methylcatechol, interleukin-6, insulin growth factor, neural growth factor, vitamin E, glutathione and leukemia inhibitory factor. The subject also may exhibit a stress to hematopoiesis. The subject may have received cancer treatment in the form of chemotherapy or radiotherapy. The subject may exhibit diabetic neuropathy. The subject may be a human. In other embodiments of either of the above methods, the neuroprotective agent is selected from the group consisting of an inhibitor of a glutamate carboxypeptidase, a eukaryotic growth factor, an inhibitor of p53, an agonist of a Trk receptor, an agonist of an RET receptor, and a Glial-Derived Neurotrophic Factor family member.

In some embodiments of either of the above methods, the agent is targeted to a site of hematopoiesis. In certain embodiments, the agent does not directly contact brain tissue. Some embodiments are characterized in that the agent is unable to restore detectable motor nerve function. In some embodiments, the agent is targeted to bone marrow. Embodiments are contemplated wherein the agent is administered in a targeting vehicle, such as a targeting vehicle is selected from the group consisting of a thixotropic gel, a liposome comprising a targeting moiety, an inclusion complex, a micelle and a fused targeting peptide. Also, the agent may be contained in a liquid solution, a suspension, an emulsion, a gel, a tablet, a pill, a capsule, a powder, a suppository, a liposome, a microparticle and a microcapsule. In any of these forms, the agent may be contained in an immediate release formulation, a controlled release formulation, a sustained release formulation, an extended release formulation, a delayed release formulation and a bi-phasic release formulation. In some embodiments, the effective amount of the agent is unable to induce regeneration of detectable sympathetic nerve fibers in the bone marrow.

Another aspect of the disclosure is drawn to a method of improving the mobilization of hematopoietic stem cells in a cancer patient comprising administering a therapeutically effective amount of a sympathetic nervous system neuroprotective agent. The method is particularly advantageous for cancer patients that have received some radio- or chemotherapy and exhibit reduced capacity for hematopoietic regeneration, limiting the numbers of mobilized HSCs obtainable from the blood for use in bone marrow transplantation following a round of systemic anti-cancer therapy.

Data disclosed herein establish that drug-induced neuropathy in the bone marrow is an important lesion preventing hematopoietic regeneration.

Particular aspects and embodiments of the disclosure are described in the following enumerated paragraphs.

1. A method of promoting hematopoietic regeneration in a subject comprising administering an effective amount of a sympathetic nervous system neuroprotective agent.

2. A method of reducing a loss of hematopoietic regeneration capacity in a subject comprising administering an effective amount of a sympathetic nervous system neuroprotective agent.

3. The method according to paragraph 1 or paragraph 2 wherein the neuroprotective agent is selected from the group consisting of 4-methylcatechol (4-MC), Glial cell-Derived Neurotrophic Factor, Glial cell-Derived Neurotrophic Factor fusion protein, interleukin-6, insulin growth factor, neural growth factor, vitamin E, glutathione leukemia inhibitory factor, acetylcysteine, acetyl-L-carnitine, amifostine, glutathione, oxcarbazepine, E2072, 2-(Phosphonomethyl) pentanedioic acid, 2-(3-mercaptopropyl)pentanedioic acid, Trypanosoma cruzi trans-sialidase/parasite-derived neurotrophic factor, Brain-Derived Neurotrophic Factor, Transforming Growth Factor-β, cardiotrophin-1, Insulin-like Growth Factor-1, basic Fibroblast Growth Factor, Vascular Endothelial Growth Factor, Hepatocyte Growth Factor Neurotrophin 3, Neurotrophin 4/5, platelet-rich plasma, pifithrin, Z-1-117, 2-imino-2,3,4,5,6,7-hexahydrobenzothiazole derivatives, 2-imino-2,3,4,5,6,7-hexahydrobenzoxazole derivatives, Gambogic amide, amitriptyline, 7,8-dihydroxyflavone, neurturin, artemin, and persephinm.

4. The method according to paragraph 3 wherein the neuroprotective agent is selected from the group consisting of Glial Cell-Derived Neurotrophic Factor, a Glial Cell-Derived Neurotrophic Factor fusion protein, 4-methylcatechol, interleukin-6, insulin growth factor, neural growth factor, vitamin E, glutathione and leukemia inhibitory factor.

5. The method according to paragraph 1 or paragraph 2 wherein the neuroprotective agent is selected from the group consisting of an inhibitor of a glutamate carboxypeptidase, a eukaryotic growth factor, an inhibitor of p53, an agonist of a Trk receptor, an agonist of an RET receptor, and a Glial-Derived Neurotrophic Factor family member.

6. The method according to paragraph 1 or paragraph 2 wherein the subject exhibits a stress to hematopoiesis.

7. The method according to paragraph 1 or paragraph 2 wherein the subject has received cancer treatment in the form of chemotherapy or radiotherapy.

8. The method according to paragraph 1 or paragraph 2 wherein the subject exhibits diabetic neuropathy.

9. The method according to paragraph 1 or paragraph 2 wherein the subject is a human.

10. The method according to paragraph 1 or paragraph 2 wherein the agent is targeted to a site of hematopoiesis.

11. The method according to paragraph 1 or paragraph 2 wherein the agent does not directly contact brain tissue.

12. The method according to paragraph 1 or paragraph 2 wherein the agent is unable to restore detectable motor nerve function.

13. The method according to paragraph 8 wherein the agent is targeted to bone marrow.

14. The method according to paragraph 1 or paragraph 2 wherein the agent is administered in a targeting vehicle.

15. The method according to paragraph 14 wherein the targeting vehicle is selected from the group consisting of a thixotropic gel, a liposome comprising a targeting moiety, an inclusion complex, a micelle and a fused targeting peptide.

16. The method according to paragraph 14 wherein the agent is contained in a liquid solution, a suspension, an emulsion, a gel, a tablet, a pill, a capsule, a powder, a suppository, a liposome, a microparticle and a microcapsule.

17. The method according to paragraph 16 wherein the agent is contained in an immediate release formulation, a controlled release formulation, a sustained release formulation, an extended release formulation, a delayed release formulation and a bi-phasic release formulation.

18. The method according to paragraph 1 or paragraph 2 wherein the effective amount of the agent is unable to induce regeneration of detectable sympathetic nerve fibers in the bone marrow.

19. A method of improving the mobilization of hematopoietic stem cells in a cancer patient comprising administering a therapeutically effective amount of a sympathetic nervous system neuroprotective agent.

Other features and advantages of the disclosure will be better understood by reference to the following detailed description, including the drawing and the examples.

### BRIEF DESCRIPTION OF THE DRAWING

FIG. 1. Cisplatin therapy induces peripheral neuropathy and reduces BM engraftment after transplantation. (A) Increased sensory neuropathy in mice treated with cisplatin (n=11) compared to saline control (n=15). (B) Experimental design to determine the effect of cisplatin on BM regeneration after transplantation. (C) Survival of saline (n=15) or cisplatin-treated (n=15) mice transplanted as described in B. (D-F) Cell counts per femoral bone marrow in saline (Sal; n=5) or cisplatin-treated (Cis; n=7) mice in protocol described in B. (D) Number of bone marrow nucleated cells (BMNC), (E) colony-forming units in culture (CFU-C), and (F) Lin⁻Sca1⁺c-kit⁺flt3⁻ cells (LSKflt3⁻) per femur. (G) Representative immunofluorescence staining to detect the presence of TH⁺ fibers in the BM; red, TH; blue, DAPI. Scale bars represent 40 μm. (H) Quantification of TH⁺ fibers in the BM of saline (n=4) or cisplatin-treated (n=6) mice analyzed as described in B.

FIG. 2. The SNS controls BM regeneration. (A) Experimental design to determine the effect of 60HDA-induced SNS lesion on BM regeneration after transplantation. (B) Survival of saline (n=25) or 60HDA-treated (n=34) mice transplanted using protocol depicted in A. (C-E) Cell counts in bone marrow. Number of (C) BMNCs, (D) CFU-C and (E) Lin⁻Sca1⁺c-kit⁺flt3⁻ cells in the BM of saline (n=7) or 60HDA-treated (n=8) transplanted mice. (F) Experimental design to determine the effect of 60HDA-induced SNS lesion on BM regeneration after 5FU injection. (G) Reduced survival of 60HDA-sympathectomized mice (n=29), compared to saline-treated controls (n=17) following 5FU. (H-J) Number of BMNC(H), CFU-C (I) and (J) Lin⁻Sca1⁺c-kit⁺flt3⁻ cells in mice treated with saline (blue; n=5-26) or 60HDA (red; n=7-28) at days 0, 4, 8 and 12 after 5FU injection. (K) Experimental design to determine the contribution of β2 and β3 adrenergic receptors to BM regeneration after 5FU injection. (L-N) Hematopoietic cell counts in BM. (L) Number of BMNC, (M) CFU-C, and (N) Lin⁻Sca1⁺c-kit⁺flt3⁻ cells per femur. A 60HDA-treated group was included as internal control. Sal, saline; SR, SR59230A. Wild type (wt)+saline (n=13); wt+SR59230A (n=5); Adrb2^{-/-}+saline (n=5); Adrb2^{-/-}+SR59230A (n=5); 60H (n=14).

FIG. 3. Bone marrow neuropathy impairs progenitor mobilization. (A) Experimental design to determine whether cisplatin treatment prevents mobilization. (B) Progenitor counts in blood after G-CSF-induced mobilization in cisplatin-treated (n=9) or saline-treated (n=10) mice. (C-D) Number of (C) CFU-C, and (D) Lin⁻Sca1⁺c-kit⁺flt3⁻cells in the bone marrow of saline (n=10) or cisplatin-treated (n=9) mice, 4 weeks after the last cisplatin injection and the G-CSF-induced mobilization protocol indicated in A. (E) Experimental design to assess if the mobilization defect in cisplatin-treated mice originates from the microenvironment. (F) Progenitor counts in blood after G-CSF-induced mobilization in saline-treated (n=9) or cisplatin-treated (n=7) mice transplanted with fresh HSC/progenitor cells and mobilized on week 37, as indicated in E.

FIG. 4 Neuroprotection restores normal BM engraftment and mobilization. (A) Experimental design to determine whether 4-methylcatechol (4-MC) induces neuroprotection from cisplatin and accelerates recovery after bone marrow transplantation. (B) 4-MC treatment reduced sensory neuropathy as determined by a nociception assay; (n=7-10 mice per group) (C) Quantification of TH+fibers and (D) representative TH immunofluorescence staining; red:TH, blue:DAPI (n=4-6 mice per group). Scale bars equal 40 μm. (E-G) 4-MC administration to cisplatin-treated mice significantly improves hematopoietic cell counts in the bone marrow after transplantation (E) Number of BMNC; (F) CFU-C; (G) Lin⁻Sca1⁺c-kit⁺flt3⁻ cells per femur; (n=5-7 mice per group). (H) Overall survival of mice after transplantation. 4-MC treatment completely prevented neuropathy-induced death from bone marrow aplasia; (n=8-15). (I) 4-MC also protected 60HDA-lesioned mice from 5FU-induced death (see FIG. 7A for experimental design); (n=8-29) (J-L) Bone marrow hematopoietic cell counts 12 days after 5FU. (J) Number of BMNC, (K) CFU-C, and (L) Lin Sca1⁺c-kit⁺flt3⁻ cells in mice treated as indicated in FIG. 7A; (n=8-15). (M and N) G-CSF-induced progenitor mobilization is restored by neuroprotection with (M) 4-MC or (N) GDNF-Fc. Saline, n=10; saline+4MC, n=5; saline+GDNF-Fc, n=5; cisplatin, n=10; cisplatin+MC, n=5; and cisplatin+GDNF-Fc, n=3.

FIG. 5. Cisplatin treatment does not affect HSC numbers in the BM. (A) Experimental design to determine the effect of cisplatin on steady-state hematopoiesis. Number of (B) bone marrow nucleated cells, (C) CFU-C, and (D) Lin⁻Sca1⁺rc-kit⁺ cells in saline (n=5) or cisplatin-treated (n=7) mice, 4 weeks after the last cisplatin injection.

FIG. 6. Chemical sympathectomy with 6-hydroxydopamine (60HDA) does not induce significant changes in hematopoietic stem, progenitor and differentiated cells in the bone marrow in steady state. Number of (A) bone marrow nucleated cells, (B) CFU-C, (C) Lin⁻Sca1⁺c-kit+flt3⁻cells, and (D) CD150⁺CD48⁻ cells in mice treated with saline (n=5-10) or 60HDA (n=3-13). BM analyses were performed 3 days after the last injection of 60HDA. (E) Cell cycle analysis (by measuring BrdU incorporation) in Lin⁻Sca1⁺c-kit+cells purified from the BM of mice treated with saline (n=7) or 60HDA (n=6), 3 days after the last injection of 60HDA. Apopt: Apoptotic cells.

FIG. 7. 4-methylcatechol (4-MC) protects sympathetic fibers from 60HDA-induced damage. (A) Experimental design to determine whether 4-MC protects nerve fibers from 60HDA and restores BM regeneration after 5FU injection. (B) Quantification of TH+fibers in the calvaria 12 days after 5FU injection; (n=4-5 mice per group). (C) Representative immunofluorescence staining of whole-mount calvaria with vessel (PECAM+(blue))-associated TH+nerves (white). Images corresponding to the TH channel were renormalized using a gamma value of 1.16. Scale bar, 100 μm.

FIG. 8. GDNF-Fc protects the sympathetic nervous system from cisplatin damage. (A) GDNF-Fc has biological activity: dose-response quantification of the percentage of PC12ES cells differentiated towards neurons after incubation with the indicated concentrations of GDNF-Fc for 1 week. (B) Experimental design to determine whether GDNF-Fc protects sympathetic fibers from cisplatin-induced damage in vivo and accelerates BM regeneration after transplantation. (C) Quantification of sensory neuropathy 2 weeks after transplantation. Saline, n=7; saline+GDNF-Fc, n=10; cisplatin, n=10; cisplatin+GDNF-Fc, n=10. (D) Quantification of TH⁺ fiber density in the BM of mice treated 4 weeks after transplantation. n=4-6 per group. (E) Representative immunofluorescence staining to assess TH⁺ fibers (red) in the BM (DAPI, blue). Scale bars, 40 μm Sal, saline; G, GDNF-Fc.

FIG. 9. GDNF-Fc restores normal BM engraftment in cisplatin-treated mice. Number of (A) BMNC, (B) CFU-C, and (C) Lin⁻Sca1⁺c-kit+flt3⁻ cells in mice treated as indicated in FIG. 8B, 4 weeks after transplant. Saline, n=5; saline+GDNF-Fc, n=7; cisplatin, n=7; and cisplatin+GDNF-Fc, n=7. (D). Probability of survival. Saline, n=15; saline+GDNF-Fc, n=3; cisplatin, n=15; and cisplatin+GDNF-Fc, n=3. Sal, saline; G, GDNF-Fc.

FIG. 10. GDNF-Fc restores normal BM regeneration after 5FU injection in 60HDA-lesioned mice. (A) Experimental design. (B) Probability of survival of mice following the protocol depicted in (A). Saline, n=17; saline+GDNF-Fc, n=7; 6OHDA, n=29; and 60HDA+GDNF-Fc, n=7. Number of (C) BMNC, (D) CFU-C, and (E) Lin⁻Sca1⁺c-kit⁺flt3⁻cells 12 days after 5FU injection. Saline, n=9; saline+GDNF-Fc, n=5; 6OHDA, n=15; and 60HDA+4MC, n=7. Sal, saline; G, GDNF-Fc.

FIG. 11. Experimental designs to determine whether (A) 4-MC- or (B) GDNF-Fc-induced neuroprotection restores mobilization in cisplatin-treated mice.

FIG. 12. Graph showing percent differentiation of cells in the presence of negative control (mock), glial cell-derived neurotrophic factor-Fc fusion (GDNF-Fc) or glial cell-derived neurotrophic factor-hemagluttinin fusion (GDNF-HA) for one week.

FIG. 13. 4-MC and GDNF-Fc induce sensory neuroprotection in cisplatin-treated mice. (a) 4-MC treatment reduced sensory neuropathy 2 weeks after transplantation as determined by a nociception assay; (n=7-10 mice per group). (b) Number of Lin⁻Sca1⁺c-kit⁺flt3⁻cells per femur; (n=5-7 mice per group) in the mice treated as in (a) 4 weeks after transplantation. (c) Quantification of sensory neuropathy 2 weeks after transplantation. (n=7-10 mice per group). (d) Number of Lin⁻Sca1⁺c-kit⁺flt3⁻ cells per femur; (n=5-7 mice per group) in the mice treated as in (a) 4 weeks after transplantation.

FIG. 14. Cisplatin therapy induces peripheral neuropathy and reduces BM engraftment after transplantation. (A) Experimental design to determine the effect of cisplatin on BM regeneration after transplantation. (B) Survival of saline (n=15) or cisplatin-treated (n=15) mice transplanted as described in A. (C) HE stain of the femur of a moribund, cisplatin-treated mice 8 days after transplant (D) Cell counts per femoral bone marrow in saline (Sal; n=5) or cisplatin-treated (Cis; n=7) mice in protocol described in A. (E) HE stains of the femur of saline- or cisplatin-treated mice 30 days after transplantation. (F) colony-forming units in culture (CFU-C), and (G) Lin⁻Sca1⁺c-kit⁺flt3⁻cells (LSKflt3⁻) per femur 30 days after transplantation (H) Representative immunofluorescence staining to detect the presence of TH⁺ fibers in the BM; red, TH; blue, DAPI. Scale bars represent 40 mm. (I) Quantification of TH⁺ fibers in the BM of saline (n=4) or cisplatin-treated (n=6) mice analyzed as described in B. (J) colony-forming units in culture (CFU-C), and (K) Lin⁻Sca1⁺c-kit⁺flt3⁻cells (LSKflt3⁻) per femur 30 days after transplantation in the BM of mice treated with saline, cisplatin (cis), vincristine (vin) or carboplatin (car). (L) Percentage of donor cells in blood of recipient mice 16 weeks after transplantation of 10⁵ BMNC collected from the femurs analyzed in J-K and transplanted together with 10⁵ competitor BMNC.

FIG. 15. Neuropathy-inducing chemotherapy agents delay BM recovery 3 months after bone marrow transplantation. (A) Representative immunofluorescence staining to detect the presence of TH⁺ fibers in the BM; red, TH; blue, DAPI. Scale bars represent 40 mm. (B) Quantification of TH⁺ fibers in the BM of saline, cisplatin, vincristine or carboplatin-treated mice 12 weeks after bone marrow transplant. (C) bone marrow nucleated cells, (D) CFU-C, and (E) Lin⁻Sca1⁺c-kit⁺Flt3⁻ cells in saline, cisplatin, carboplatin or vincristine-treated mice, 3 months after bone marrow transplantation of 10⁶ BMNC (n=3 mice per group).

FIG. 16. Bone marrow regeneration is complete 4 months after bone marrow transplantation (BMT) in cisplatin-treated mice. (A) Experimental design to determine the effect of cisplatin on long-term BM recovery after transplantation. (B) bone marrow nucleated cells, (C) CFU-C, and (D) Lin⁻Sca1⁺c-kit⁺Flt3⁻ cells in saline or cisplatin-treated mice, 4 months after bone marrow transplantation of 10⁶ BMNC. (E) Competitive reconstitution units in the BM of the mice analyzed in B-D.

FIG. 17. The SNS is required for BM regeneration after transplantation. (A) Experimental design to determine the effect of 60HDA-induced SNS lesion on BM regeneration after transplantation. (B) Survival of saline (n=25) or 60HDA-treated (n=34) mice transplanted using protocol depicted in A; p<0.001; Logrank test (C-E) Cell counts in bone marrow. Number of (C) BMNCs, (D) CFU-C and (E) Lin⁻Sca1⁺c-kit⁺flt3⁻ cells in the BM of saline (n=7) or 60HDA-treated (n=8) transplanted mice.

FIG. 18. No defect in HSPC homing efficiency after 60HDA or cisplatin treatment. Percentage of donor CFU-C detected in the BM of (A) saline- (blue) or 60HDA- (red) and (B) saline- (black) or cisplatin- (grey) mice 24 hours after lethal irradiation (1200 rads) and injection of 5×10⁶ donor BMNC.

FIG. 19. The SNS controls BM recovery. (A) Experimental design to determine the effect of 60HDA-induced SNS lesion on BM regeneration after 5FU challenge. (B) Reduced survival of 60HDA-sympathectomized mice (n=29), compared to saline-treated controls (n=17) following 5FU. (C-E) Number of BMNC(C), CFU-C (D) and (E) Lin⁻Sca1⁺c-kit⁺flt3⁻ cells in mice treated with saline (blue; n=5-26) or 60HDA (red; n=7-28) at days 0, 4, 8 and 12 after 5FU injection. (F-G) Percentage of proliferating (F) and viable (G) LSK cells in the BM of saline or 60HDA-treated mice, 8 days after 5FU challenge. (H) Number of BMNC, (I) CFU-C, and (J) Lin⁻Sca1⁺c-kit⁺flt3⁻ cells per femur in the BM of WT or TH-Cre:iDTR mice after DT and 5FU injection. (K) Representative whole-mount immunofluorescence staining to detect the presence of TH⁺ fibers in the sternum of WT or TH-Cre:iDTR mice 12 days after 5FU injection. (L) Number of BMNC, (M) CFU-C, and (N) Lin⁻Sca1⁺c-kit⁺flt3⁻ cells per femur in the BM of control or TH-Cre:p53^{flox/flax} mice treated with saline or cisplatin 30 days after transplantation. (O) Representative immunofluorescence stain of the BM of a cisplatin-treated TH-Cre: p53^{flox/flax} mice 30 days after transplantation (P). Quantification of TH⁺ fibers in the BM of the mice analyzed in I-K. (O) Number of BMNC, (R) CFU-C, and (S) Lin⁻Sca1⁺c-kit⁺flt3⁻ cells per femur in the BM of WT or Adrb2^{-/-} treated with saline, SR59230A (SR) or ICI118 551 (ICI), A 60HDA-treated group was included as internal control. (T) Representative immunofluorescence stain of the BM of saline or 60HDA-treated Nestin-gfp mice showing reduction in BM cellularity (note vessel enlargement) and no variation in endothelial cells (CD31 red). (U) Number of Nestin+cells per femur in saline or 60HDA-treated Nestin-gfp mice prior or 12 days after 5FU injection. (V) Percentage of viable Nestin⁺ cells in the BM of saline or 60HDA-treated Nestin-gfp mice 24 hours after 5FU injection.

FIG. 20. Sympathetic nerve damage and not 5FU neurotoxicity prevents BM regeneration. (A) Representative immunofluorescence staining for TH+sympathetic nerve fibers in the calvaria BM of saline or 5-Fu injected (250 mg/kg) mice 48 h after 5-Fu injection. (B) Quantification of TH⁺ fibers in the calvaria 48 h after 5-Fu injection. (C) Experimental design to determine whether sublethal irradiation of 60HDA-treated mice also results in reduced BM recovery. Number of (D) bone marrow nucleated cells, (E) CFU-C, (F) Lin⁻Sca1⁺c-kit⁺flt3⁻ cells regeneration. BM analyses were performed 12 days after irradiation.

FIG. 21. Niche analyses in saline or 60HDA sympathectomized mice. (A) Representative immunofluorescence staining for Nestin-gfp (green) endothelial cells (PECAM-1; red), monocyte macrophages (CD68+, white) or (B) Perivascular α-SMA+cells (white) and endothelial cells (PECAM-1; red) or (C) osteoblasts and endothelial cells (PECAM-1; red) in the BM of saline or 60HDA treated mice prior 5FU injection. Percentage of macrophages (D) and endothelial cells (E) per femur or osteoblasts in bone (F) in the BM of saline or 60HDA treated mice prior 5FU injection. (G-H) As A-B but 12 days after 5FU injection. (1-K) As D-E but 12 days after 5FU injection.

FIG. 22. Neuroprotection restores normal BM engraftment and mobilization. (A) Experimental design to determine whether 4-methylcatechol (4-MC) induces neuroprotection from 60HDA and accelerates recovery after bone marrow transplantation. (B) Overall survival of saline or 60HDA-treated mice after 4-MC neuroprotection and 5FU injection. (C) LSKF cells per femur in mice treated as indicated in A, 12 days after 5FU injection. (D) Quantification of TH⁺ fibers in the calvaria 12 days after 5FU injection; (n=4-5 mice per group). (E) Representative immunofluorescence staining of whole-mount calvaria with vessel (PECAM⁺(blue))-associated TH⁺ nerves (white). Images corresponding to the TH channel were renormalized using a gamma value of 1.16. Scale bar, 100 mm. Percentage of Nestin cells per femur in Nestin-gfp mice treated as indicated in A prior (F) or 12 days after (G) 5FU injection. (H) Representative immunofluorescence staining of Nestin-gfp mice treated with 60HDA (left) or 60HDA+4-MC mice (right); red:PECAM, white:DAPI, green: Nestin cells. (I) Scheme of TrkA receptor expressor in Ta1-Cre:TrkA^{Neo/Neo} mice. Number of BMNC (I), CFU-C (J), and Lin⁻Sca1⁺c-kit⁺flt3⁻ cells (K) in WT or Ta1-Cre:TrkA^{Neo/Neo} mice treated with 60HDA and 4-MC 12 days after 5FU injection. Overall survival (L) and number of BMNC (M), CFU-C(N), and Lin⁻Sca1⁺c-kit⁺flt3⁻ cells per femur 30 days after transplant (O) in saline or cisplatin mice after 4-MC neuroprotection and transplantation. (P) Quantification of TH⁺ fibers in the BM of the mice analyzed in N-P. (O) Nestin⁺ cells per femur in the BM of saline or cisplatin-treated mice after 4-MC neuroprotection prior transplantation. (R) G-CSF-induced progenitor mobilization is restored by neuroprotection with 4-MC or GDNF-Fc.

FIG. 23. Niche analyses in saline or 60HDA-treated mice after 4-MC neuroprotection. Number of BMNC (A) and CFU-C (B) in mice treated as indicated in FIG. 22A, 12 days after 5FU injection. (C) Percentage of donor cells in blood of recipient mice 16 weeks after transplantation of 10⁵ BMNC collected from the femurs analyzed in A-B and transplanted together with 10⁵ competitor BMNC. Percentage of macrophages (D) and endothelial cells (E) per femur or osteoblasts in bone (F) in the BM of saline or 60HDA treated mice after 4-MC neuroprotection and prior 5FU injection. (G-I) As D-F but 12 days after 5FU injection. (J-K) AsGF-H but 12 days after 5FU injection.

FIG. 24. Niche analyses in saline or cisplatin-treated mice after 4-MC neuroprotection. (A) Number of LTC-IC per femur in the BM of mice analyzed in FIG. 22N-P. (B) Percentage of donor cells in blood of recipient mice 16 weeks after transplantation of 10⁵ BMNC collected from the femurs analyzed in N-P and transplanted together with 10⁵ competitor BMNC. (C) Representative immunofluorescence staining of TH-fibers in the BM of the mice analyzed in FIG. 22N-P. Percentage of macrophages (D) per femur or osteoblasts in bone (E) in the BM of saline or cisplatin treated mice after 4-MC neuroprotection and prior transplantation.

### DETAILED DESCRIPTION

Hematopoietic defects resulting from anti-cancer agents are caused by damage to adrenergic nerve fibers that innervate the bone marrow. Furthermore, neuro-regenerative therapy using 4-methylcatechol or glial-derived neurotrophic factor (GDNF) restored hematopoietic recovery and progenitor mobilization. Thus, adrenergic signals critically contribute to bone marrow regeneration. The data disclosed herein establish the benefit of neuroprotection to shield hematopoietic niches.

The disclosure provides methods for preventing degeneration of hematopoietic capacity and methods for promoting or inducing hematopoietic regeneration comprising administration of a prophylactically or therapeutically useful amount of a neuroprotective agent (i.e., an anti-neuropathic agent). Disclosed herein in support are data identifying bone marrow neuropathy as a critical stromal lesion compromising hematopoietic regeneration after cytotoxic chemotherapy. Evidence is provided that adrenergic signals transmitted by both the β2 and β3 adrenoreceptors allow HSCs to respond appropriately to hematopoietic stress, balancing proliferation and differentiation to replenish the bone marrow compartment and peripheral blood cells. Without adrenergic signals, HSCs fail to proliferate, leading to increased mortality from bone marrow aplasia. Nerves and perivascular stromal cells appear functionally associated in BM as neuro-reticular complexes where nestin⁺mesenchymal stem cells have been recently suggested to form HSC niches. The number of nestin⁺niche cells, however, was not altered in sympathectomized 5FU-treated mice, revealing that HSC niches are present but unable to support regeneration without adrenergic input.

The current studies provide the proof-of-principle that HSC expansion and the response to mobilization in animals subjected to hematopoietic stress are aided by co-administration of a neuroprotective agent whenever cytotoxic treatments, such as chemo- or radio-therapy treatments of cancer, are administered. Neuroprotective agents coupled with conventional cytotoxic therapy (e.g., chemotherapy) are expected to limit chronic myelotoxicity and provide additional therapeutic options to previously treated cancer patients and others receiving cytotoxins.

The following general description of aspects of the disclosure provide additional description and teachings of subject matter of the disclosure, followed by working examples of that subject matter.

### Disease States

Diseases (or disorders or conditions) associated with a degradation or decrease in hematopoiesis include the diseases/disorders/conditions apparent from Table 1. Inspection of Table 1 reveals that any of a number of toxins can lead to, or be associated with, various neuropathies. All such conditions, including but not limited to peripheral sympathetic sensory neuropathies, are contemplated as diseases/disorders/conditions associated with a degradation in hematopoietic capacity that would benefit from prophylactic or therapeutic administration of the agents according to the disclosure.

TABLE 1
Toxic Neuropathies

| | | |
|---|---|---|
| Circumstances of Toxicity | Neuropathy | Comments |

Axonopathy

| | | | |
|---|---|---|---|
| Nonpharmaceutical toxins | | | |
| Acrylamide monomer | Flocculators, grouting agents | Sensory ataxia; large | Numbness, excessive |
| | | fiber | sweating, exfoliative |
| | | | dermatitis |
| Allyl chloride | Epoxy resin, glycerin | Dysesthesia and distal | |
| | | weakness | |
| Arsenic (inorganic) | Copper/lead smelting, contaminant | S > M; painful; usually | Skin: hyperkeratosis, “rain- |
| | in recreational drugs, | subacute or chronic; | drop” pigmentation of skin, |
| | suicide/homicide | may be acute following | Mees' line in nails |
| | (herbicide/insecticide) | large doses | |
| Carbon disulphide | Viscose rayon, cellophane; airborne | SM | Slow NCS |
| | industrial exposure | | |
| Dimethylaminopropionitrile | Polyurethane foam | SM | Small-fiber neuropathy |
| (DMAPN) | | | with prominent bladder |
| | | | symptoms and impotence |
| Ethylene oxide | Sterilization of biomedicals | | |
| Hexacarbons (paranodal | Solvents, adhesives | SM | Neurofilament swelling of |
| giant axonal) | Substance abuse (glues and | | axons; CNS |
| | thinners) | | |
| Lead | Batteries, smelting metal ores, | M > S; wrist drop | Burton's line, anemia, |
| | paints | | basophilic stippling |
| Mercury (inorganic) | Environmental/workplace | CNS > PNS; neuropathy | Tremor, insomnia, |
| | | uncommon | behavioral change |
| Methyl bromide | Fumigant, insecticide, refrigerant, | Variable recovery | Encephalitis, ataxia |
| | fire extinguisher | | |
| Organophosphorus esters | Insecticide, petroleum, plastics | SM | Acute toxicity presents as |
| | | | cholinergic crisis |
| Thallium (rat poison) | Rodenticides, insecticides | Painful SM | Thallium (alopecia, Mees' |
| | | | line, hyperkeratosis) |
| Vacor | Rodenticide, suicide | Rapid onset of severe | Diabetic ketoacidosis a |
| | | axonopathy and | feature of acute toxicity |
| | | autonomic dysfunction | |
| Pharmaceutical agents | | | |
| Chloramphenicol | Mean cumulative dose 255 g, | S > M | Also optic neuropathy |
| | duration | | |
| Colchicine | Chronic dosing at 1.2 mg/d | Distal paresthesias and | Also myopathy with |
| | especially in the presence of renal | proximal weakness | elevated serum CK |
| | dysfunction | | |
| Dapsone | 200-400 mg/d over many months | Pure motor, especially | May look like motor |
| | | upper limbs | neuron disease |
| Disulfiram | 250-500 mg/d after several months | SM | Difficult to distinguish |
| | used for alcoholism | | from alcohol neuropathy |
| Ethambutol | >20 mg/kg per day over many | Sensory neuropathy | Also optic neuropathy |
| | months | | |
| Ethionamide | >15 mg/kg | Sensory neuropathy | Limited by GL dermatologic |
| | | | and CNS side effects |
| Gold | Controversial, as | S > M with myokymia | Rash, pruritus |
| | rheumatoid arthritis can | | |
| | cause neuropathy | | |
| | Not dose dependent | | |
| Isoniazid | >5 mg/kg over weeks or about 6 | Dose-dependent SM | Add pyridoxine 50 mg/d |
| | months, depending on acetylator | neuropathy | when using INH |
| | status | | |
| Metronidazole | Cumulative dose >30 g | Sensory (small and large | |
| | | fiber) | |
| Misonidazole | Cumulative dose >18 g/m² | Sensory axonopathy | Dose-limiting side effect |
| Nitrofurantoin | Standard dose of 200 mg/day over a | Mild SM neuropathy | |
| | few weeks | | |
| Nitrous oxide | Dental surgery, anesthesia, | S >> M | Toxic myeloneuropathy |
| | substance abuse | | resembles cobalamine |
| | | | deficiency |
| Nucleoside analogues | >12.5 mg/kg per day for ddI, 0.02 | Painful sensory | Difficult to distinguish |
| (ddC, ddI, 4dT) | mg/kg per day for ddC and 0.5 | neuropathy | from HIV neuropathy |
| | mg/kg per day for 4dT | | |
| Pyridoxine | >200 mg a day over several months | Length-dependent | Neuronopathy at higher |
| | | axonopathy | doses |
| Suramin | Peak serum concentration of | S > M: may be | |
| | 350 μg/mL | demyelinating | |
| Taxol | Cumulative dose of >1500 mg/m² | S > M | Higher single doses may |
| | | | cause neuronopathy |
| Thalidomide | 100 mg/d for 6 months. | S > M | Thalidomide (brittle nails, |
| | | | palmar erythema) |
| Vincristine and other vinca | Almost all patients | S > M but autonomic | Vacuolar myopathy |
| alkaloids | | fibers also affected | |

Myelinopathy

| | | | |
|---|---|---|---|
| Amiodarone | 400 mg/day for 6-36 months, | SM; dose-dependent | Tremor |
| | serum concentration of 2.4 mg/L | | |
| Perhexiline | Not dose-related | S (large fiber) and M, | Hepatic toxicity |
| | | facial, autonomic | |
| Polychlorinated biphenyls | Plasticizers, electrical insulators | SM | Acne, brown nails |
| Suramin | Not dose-related | Demyelinating like | |
| | | subacute GBS | |
| Trichloroethylene | Dry-cleaning, rubber, degreasing | Mainly cranial nerves: | Limbs rarely affected |
| | agent | trigeminal, facial, | |
| | | oculomotor, optic | |

Sensory Neuronopathy

| | | | |
|---|---|---|---|
| Platinum compounds, e.g., | Cumulative dose more than 900 mg/m² | Large-fiber sensory | Irreversible |
| cisplatin | | | |
| High-dose pyridoxine | Massive parenteral doses in grams | Sensory neuronopathy; | May be irreversible |
| | over days | gait ataxia, | |
| | | pseudoathetosis | |
| Taxol | Single dose of ≧250 mg/m² | Sensory ataxia | May be irreversible |

### Cytotoxic Agents

Many of the cytotoxins known in the art and/or disclosed in Table 1 are known to be useful in cancer therapy and, in fact, the disclosure contemplates neuropathies associated with prior chemotherapy of any kind, including chemotherapy with a platinum-based anti-cancer agent such as cisplatin. Moreover, the disclosure contemplates injuries to hematopoietic stem cell proliferation or mobilization, collectively hematopoietic capacity, by any chemical or physical agent, such as any chemotherapeutic or any form of radiation therapy, to produce a subject that is amenable to the treatment methods of the instant disclosure. The prophylactic methods according to the disclosure are amenable to the pre-treatment of subjects, such as human cancer patients, prior to undergoing cancer radio- or chemotherapy.

### Neuroprotective Agents or Anti-Neuropathic Agents

The disclosure establishes that neuroprotective agents, or anti-neuropathic agents, are useful in hematopoietic recovery, bone marrow regeneration and progenitor cell mobilization following exposure of an organism to a physical or chemical stress, such as radio- or chemo-therapy to treat cancer. Any compound known in the art is contemplated as useful in the methods of preventing, treating or ameliorating a symptom associated with loss or reduction of hematopoiesis, mobilization of progenitor cells, particularly from the bone marrow, or repopulation of bone marrow niches following cell loss. Exemplary compounds useful in such methods include, but are not limited to, 4-methylcatechol (4-MC), Glial cell-Derived Neurotrophic Factor (GDNF), Glial cell-Derived Neurotrophic Factor fusion protein, interleukin-6, insulin growth factor, neural growth factor, vitamin E, glutathione and leukemia inhibitory factor. In addition, the following compounds are useful in the methods disclosed herein.

Acetylcysteine (N-acetylcysteine, NAC) has been the subject of several studies that indicate that this compound induces neuroprotection or nerve regeneration. See Hart, et al., Sensory neuroprotection, mitochondrial preservation, and therapeutic potential of N-acetyl-cysteine after nerve injury. Neuroscience, 2004. 125(1): p. 91-101; Lin, et al., N-acetylcysteine has neuroprotective effects against oxaliplatin-based adjuvant chemotherapy in colon cancer patients: preliminary data. Support Care Cancer, 2006. 14(5): p. 484-7. Each of the two references is specifically incorporated by reference herein.

Acetyl-L-carnitine also is known to induce neuroprotection. See McKay Hart, et al., Pharmacological enhancement of peripheral nerve regeneration in the rat by systemic acetyl-L-carnitine treatment. Neurosci Lett, 2002. 334(3): p. 181-5; Sima, A. A., et al., Acetyl-L-carnitine improves pain, nerve regeneration, and vibratory perception in patients with chronic diabetic neuropathy: an analysis of two randomized placebo-controlled trials. Diabetes Care, 2005. 28(1): p. 89-94. Each of the two references is specifically incorporated by reference herein.

Amifostine is another compound believed to protect from chemotherapy-induced neuropathy. See Hilpert, et al., Neuroprotection with amifostine in the first-line treatment of advanced ovarian cancer with carboplatin/paclitaxel-based chemotherapy—a double-blind, placebo-controlled, randomized phase II study from the Arbeitsgemeinschaft Gynakologische Onkologoie (AGO) Ovarian Cancer Study Group. Support Care Cancer, 2005. 13(10): p. 797-805; Kanat, et al., Protective effect of amifostine against toxicity of paclitaxel and carboplatin in non-small cell lung cancer: a single center randomized study. Med Oncol, 2003. 20(3): p. 237-45. Each of the two references is specifically incorporated by reference herein.

Glutathione (GSH) has been reported as a compound that prevents platinum accumulation. See Cascinu, et al., Neuroprotective effect of reduced glutathione on cisplatin-based chemotherapy in advanced gastric cancer: a randomized double-blind placebo-controlled trial. J Clin Oncol, 1995. 13(1): p. 26-32; Cascinu, et al., Neuroprotective effect of reduced glutathione on oxaliplatin-based chemotherapy in advanced colorectal cancer: a randomized, double-blind, placebo-controlled trial. J Clin Oncol, 2002. 20(16): p. 3478-83; Milla, et al., Administration of reduced glutathione in FOLFOX4 adjuvant treatment for colorectal cancer: effect on oxaliplatin pharmacokinetics, Pt-DNA adduct formation, and neurotoxicity. Anticancer Drugs, 2009. 20(5): p. 396-402. Each of the three references is specifically incorporated by reference herein.

Oxcarbazepine (OXC) can also induce neuroprotection from chemotherapy. See Argyriou, et al., Efficacy of oxcarbazepine for prophylaxis against cumulative oxaliplatin-induced neuropathy. Neurology, 2006. 67(12): p. 2253-5. The reference is specifically incorporated by reference herein.

Inhibitors of glutamate carboxypeptidase, such as E2072, which is a compound known to inhibit glutamate carboxypeptidase and to induce neuroprotection in rats. See Carozzi, et al., Glutamate carboxypeptidase inhibition reduces the severity of chemotherapy-induced peripheral neurotoxicity in rat. Neurotox Res, 2010. 17(4): p. 380-91, incorporated by reference herein.

2-(Phosphonomethyl) pentanedioic acid (2-PMPA) and 2-(3-mercaptopropyl)pentanedioic acid (2-MPPA) also each inhibit glutamate carboxypeptidase. See Thomas, et al., Glutamate carboxypeptidase II (NAALADase) inhibition as a novel therapeutic strategy. Adv Exp Med Biol, 2006. 576: p. 327-37; discussion 361-3; Zhang, et al., The preventive and therapeutic effects of GCPII (NAALADase) inhibition on painful and sensory diabetic neuropathy. J Neurol Sci, 2006. 247(2): p. 217-23. Each of the two references is specifically incorporated by reference herein.

Trypanosoma cruzi trans-sialidase/parasite-derived neurotrophic factor (PDNF) promotes neuronal survival through Trk receptors, thereby functioning as a neuroprotective agent or anti-neuropathic agent. See Chuenkova, et al., Trypanosoma cruzi-Derived Neurotrophic Factor: Role in Neural Repair and Neuroprotection. J Neuroparasitology, 2010. 1: p. 55-60, incorporated by reference herein.

In addition to the foregoing exemplary compounds, a variety of growth factors, e.g., eukaryotic cell growth factors, function as neuroprotective agents or anti-neuropathic agents. Growth factors such as Brain-Derived Neurotrophic Factor (BDNF) and Transforming Growth Factor-β (TGF-β) [Sakamoto, et al., Adenoviral gene transfer of GDNF, BDNF and TGF beta 2, but not CNTF, cardiotrophin-1 or IGF1, protects injured adult motoneurons after facial nerve avulsion. J Neurosci Res, 2003. 72(1): p. 54-64], cardiotrophin-1 (CT-1) and Insulin-like Growth Factor-1 (IGF-1) [Rind, et al., Target-derived cardiotrophin-1 and insulin-like growth factor-I promote neurite growth and survival of developing oculomotor neurons. Mol Cell Neurosci, 2002. 19(1): p. 58-71], basic Fibroblast Growth Factor (bFGF) [Jungnickel, et al., Faster nerve regeneration after sciatic nerve injury in mice over-expressing basic fibroblast growth factor. J Neurobiol, 2006. 66(9): p. 940-8; Grothe, et al., Physiological function and putative therapeutic impact of the FGF-2 system in peripheral nerve regeneration—lessons from in vivo studies in mice and rats. Brain Res Rev, 2006. 51(2): p. 293-9], Vascular Endothelial Growth Factor (VEGF) [Yu, et al., Vascular endothelial growth factor mediates corneal nerve repair. Invest Ophthalmol V is Sci, 2008. 49(9): p. 3870-8]; Hepatocyte Growth Factor (HGF) [Tonges, et al., Hepatocyte growth factor protects retinal ganglion cells by increasing neuronal survival and axonal regeneration in vitro and in vivo. J Neurochem, 2011. 117(5): p. 892-903]; and Neurotrophins 3 and 4/5 [Tabakman, et al., Interactions between the cells of the immune and nervous system: neurotrophins as neuroprotection mediators in CNS injury. Prog Brain Res, 2004. 146: p. 387-401]. Each of the references cited in this paragraph is incorporated by reference herein.

Platelet-rich plasma, which is rich in growth factors [Yu, et al., Platelet-rich plasma: a promising product for treatment of peripheral nerve regeneration after nerve injury. Int J Neurosci, 2011. 121(4): p. 176-80], incorporated by reference herein.

Inhibitors of p53 function, such as pifithrin- (PFT) and Z-1-117, as well as other p53 inhibitors expressly identified in Zhu, et al., Novel p53 inactivators with neuroprotective action: syntheses and pharmacological evaluation of 2-imino-2,3,4,5,6,7-hexahydrobenzothiazole and 2-imino-2,3,4,5,6,7-hexahydrobenzoxazole derivatives. J Med Chem, 2002. 45(23): p. 5090-7, incorporated by reference herein.

Additional categories of compounds suitable for the methods disclosed herein include, but are not limited to, Trk receptor(s) agonists, such as Gambogic amide [Jang, et al., Gambogic amide, a selective agonist for TrkA receptor that possesses robust neurotrophic activity, prevents neuronal cell death. Proc Natl Acad Sci USA, 2007. 104(41): p. 16329-34], Amitriptyline [Jang, et al., Amitriptyline is a TrkA and TrkB receptor agonist that promotes TrkA/TrkB heterodimerization and has potent neurotrophic activity. Chem Biol, 2009. 16(6): p. 644-56], 7,8-Dihydroxyflavone [Jang, et al., A selective TrkB agonist with potent neurotrophic activities by 7,8-dihydroxyflavone. Proc Natl Acad Sci USA, 2010. 107(6): p. 2687-92] and others expressly disclosed in Zaccaro, et al., Selective small molecule peptidomimetic ligands of TrkC and TrkA receptors afford discrete or complete neurotrophic activities. Chem Biol, 2005. 12(9): p. 1015-28. Each of these references is incorporated by reference herein.

Yet another category of compounds useful in the disclosed methods is RET receptor(s) agonists and GDNF family members like neurturin, artemin and persephinm. See Bespalov, et al., GDNF family receptor complexes are emerging drug targets. Trends Pharmacol Sci, 2007. 28(2): p. 68-74, incorporated by reference herein.

Beyond the compounds expressly disclosed herein as neuroprotective agents or anti-neuropathic agents in the context of the disclosed methods, any compound known to be neuroprotective, such as any compound known to inhibit p53 or to function as an agonist of either a Trk receptor or an RET receptor, is contemplated for use in the disclosed methods.

### Conjugates/Fusions: Targeted Forms

One of ordinary skill in the art will readily appreciate that the anti-neuropathic agents of the disclosure can be modified in any number of ways, such that the therapeutic or prophylactic efficacy of the anti-neuropathic agent is increased through the modification. For instance, the anti-neuropathic agent can be conjugated either directly or indirectly through a linker to a targeting moiety. The practice of conjugating compounds to targeting moieties is known in the art. See, e.g., Wadhwa et al., J Drug Targeting, 3, 111-127 (1995) and U.S. Pat. No. 5,087,616. The term “targeting moiety” as used herein, refers to any molecule or agent that specifically recognizes and binds to a targeting compound in vivo, such as a free targeting compound (e.g., SDF-1) or a cell-surface receptor, such that the targeting moiety directs the delivery of the anti-neuropathic agent to a locus in a body or to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., CXCR4, Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups. The term “linker” in some embodiments refers to any agent or molecule that bridges the anti-neuropathic agent to the targeting moiety. One of ordinary skill in the art recognizes that sites on the anti-neuropathic agent, which are not necessary for the function of the anti-neuropathic agent, are ideal sites for attaching a linker and/or a targeting moiety, provided that the linker and/or targeting moiety, once attached to the anti-neuropathic agent, do(es) not interfere with the function of the anti-neuropathic agent, as described herein and as exemplified by GDNF-Fc and GDNF-HA.

### Pharmaceutical Compositions and Formulations

In some embodiments, the anti-neuropathic agent, the pharmaceutically acceptable salt thereof, or the conjugate comprising the anti-neuropathic agent, is formulated into a pharmaceutical composition comprising the anti-neuropathic agent, the pharmaceutically acceptable salt thereof, or the conjugate comprising the anti-neuropathic agent, along with a pharmaceutically acceptable carrier, diluent, or excipient.

In some embodiments, the anti-neuropathic agent is present in the pharmaceutical composition at a purity level suitable for administration to a patient. In some embodiments, the anti-neuropathic agent has a purity level of at least about 90%, about 91%, about 92%, about 93%, about 94%, about 95%, about 96%, about 97%, about 98% or about 99%, and a pharmaceutically acceptable diluent, carrier or excipient.

Depending on the route of administration, the pharmaceutical composition comprising the anti-neuropathic agent may further comprise additional pharmaceutically acceptable ingredients, including, for example, acidifying agents, additives, adsorbents, aerosol propellants, air displacement agents, alkalizing agents, anti-caking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

Accordingly, in some embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC) chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, postassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In some embodiments, the foregoing component(s) may be present in the pharmaceutical composition at any concentration, such as, for example, at least A, wherein A is 0.0001% w/v, 0.001% w/v, 0.01% w/v, 0.1% w/v, 1% w/v, 2% w/v, 5% w/v, 10% w/v, 20% w/v, 30% w/v, 40% w/v, 50% w/v, 60% w/v, 70% w/v, 80% w/v, or 90% w/v. In some embodiments, the foregoing component(s) may be present in the pharmaceutical composition at any concentration, such as, for example, at most B, wherein B is 90% w/v, 80% w/v, 70% w/v, 60% w/v, 50% w/v, 40% w/v, 30% w/v, 20% w/v, 10% w/v, 5% w/v, 2% w/v, 1% w/v, 0.1% w/v, 0.001% w/v, or 0.0001%. In other embodiments, the foregoing component(s) may be present in the pharmaceutical composition at any concentration range, such as, for example from about A to about B. In some embodiments, A is 0.0001% and B is 90%.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capable of buffering at the desired pH such as, for example, phosphate buffers (e.g., PBS), triethanolamine, Tris, bicine, TAPS, tricine, HEPES, TES, MOPS, PIPES, cacodylate, MES, and others. In certain embodiments, the strength of the buffer is at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 120 mM, at least 150 mM, or at least 200 mM. In some embodiments, the strength of the buffer is no more than 300 mM (e.g., at most 200 mM, at most 100 mM, at most 90 mM, at most 80 mM, at most 70 mM, at most 60 mM, at most 50 mM, at most 40 mM, at most 30 mM, at most 20 mM, at most 10 mM, at most 5 mM, at most 1 mM).

### Routes of Administration

With regard to the disclosure, the anti-neuropathic agent, pharmaceutical composition comprising the same, conjugate comprising the same, or pharmaceutically acceptable salt thereof, may be administered to the subject by any suitable route of administration. The following discussion on routes of administration is merely provided to illustrate exemplary embodiments and should not be construed as limiting the scope of the disclosure in any way.

Formulations suitable for oral administration may consist of (a) liquid solutions, such as an effective amount of the anti-neuropathic agent of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the anti-neuropathic agent in a flavor, usually sucrose and acacia or tragacanth, as well as pastilles comprising the anti-neuropathic agent in an inert base, such as gelatin and glycerin, or sucrose and acacia, emulsions, gels, and the like containing, in addition to, such excipients as are known in the art.

The anti-neuropathic agent, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the anti-neuropathic agent is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, intrathecal, or intravenous. The anti-neuropathic agent can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations may contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the anti-neuropathic agents can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the anti-neuropathic agent can be formulated as inclusion complexes, such as cyclodextrin inclusion complexes, or liposomes.

### Dosages

The anti-neuropathic agents are useful in methods of inhibiting hematopoietic degeneration and in methods of promoting hematopoietic regeneration, as well as related conditions, as described herein. For purposes of the disclosure, the amount or dose of the anti-neuropathic agent administered should be sufficient to effect, e.g., a therapeutic or prophylactic response, in the subject or animal over a reasonable time frame. For example, the dose of the anti-neuropathic agent should be sufficient to effect a therapeutic result in a period of from about 1 to 4 hours or 1 to 4 weeks or longer, e.g., 5 to 20 or more weeks, from the time of administration. In certain embodiments, the time period could be even longer. The dose will be determined by the efficacy of the particular anti-neuropathic agent and the condition of the animal (e.g., human), as well as the body weight of the animal (e.g., human) to be treated.

Many assays for determining an administered dose are known in the art. For purposes herein, an assay, which comprises comparing the extent to which hematopoietic degeneration is treated upon administration of a given dose of the anti-neuropathic agent to a mammal among a set of mammals, each set of which is given a different dose of the anti-neuropathic agent, could be used to determine a starting dose to be administered to a mammal. The extent to which hematopoietic degeneration is treated upon administration of a certain dose can be assayed by methods known in the art, including, for instance, the methods described in the Examples set forth below.

The dose of the anti-neuropathic agent also will be determined by the existence, nature and extent of any adverse side effects that might accompany the administration of a particular anti-neuropathic agent. Typically, the attending physician will decide the dosage of the anti-neuropathic agent with which to treat each individual patient, taking into consideration a variety of factors, such as age, body weight, general health, diet, sex, cardiac metabolic modifier of the present disclosure to be administered, route of administration, and the severity of the condition being treated. By way of example and not intending to limit the invention, the dose of the anti-neuropathic agent can be about 0.000001 to about 1 g/kg body weight of the subject being treated/day, from about 0.0001 to about 0.001 g/kg body weight/day, or about 0.01 mg to about 1 g/kg body weight/day. In some embodiments, the individual dose is 10 μg/kg (e.g., 4-methylcatechol) or 250 μg/kg (e.g., Glial Cell-Derived Neurotrophic Factor). In some embodiments, the dose does not result in sensory nerve fiber growth in bone marrow that is detectable using an antibody-based staining assay as described herein.

In some embodiments, the administered dose of the anti-neuropathic agent (e.g., any of the doses described above), provides the subject with a plasma concentration of the anti-neuropathic agent of at least or about 500 nM. In some aspects, the administered dose of the anti-neuropathic agent provides the subject with a plasma concentration of the anti-neuropathic agent within a range of about 500 nM to about 2500 nM (e.g., about 750 nM to about 2000 nM, about 1000 nM to about 1500 nM). In some aspects, the dose of the anti-neuropathic agent provides the subject with a plasma concentration of the cardiac metabolic modifier which is below 100 μmol/L, e.g., below 50 μmol/L, below 25 μmol/L, below 10 μmol/L. In some embodiments, the anti-neuropathic agent delivery is targeted in a manner that renders serum concentration less relevant, for example in direct injection or infusion into a tumor or into bone.

### Controlled Release Formulations

In some embodiments, the anti-neuropathic agent described herein can be modified into a depot form, such that the manner in which the anti-neuropathic agent is released into the body to which it is administered is controlled with respect to time and location within the body (see, for example, U.S. Pat. No. 4,450,150). Depot forms of anti-neuropathic agents can be, for example, an implantable composition comprising the anti-neuropathic agents and a porous or non-porous material, such as a polymer, wherein the anti-neuropathic agent is encapsulated by or diffused throughout the material and/or degradation of the non-porous material. The depot is then implanted into the desired location within the body of the subject and the anti-neuropathic agent is released from the implant at a predetermined rate.

The pharmaceutical composition comprising the anti-neuropathic agent may be modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant compositions may further comprise, for example, micelles or liposomes, or some other encapsulated form, or may be administered in an extended release form to provide a prolonged storage and/or delivery effect. The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

In the following examples, Example 1 provides materials and methods used in the studies described herein, Example 2 discloses the use of cisplatin to induce hematopoietic degeneration, Example 3 shows the use of cisplatin to generate a sympathetic neuropathy in bone marrow, Example 4 demonstrates that 5-fluorouracil (5FU) treatment to ablate proliferating cells induced quiescent HSCs to repopulate the bone marrow in cisplatin-treated mice, Example 5 established that β2 and β3 adrenergic receptors were involved in hematopoietic regeneration, Example 6 shows that cisplatin treatment produced bone marrow neuropathy that markedly compromised HSC/progenitor trafficking, Example 7 establishes that protection from cisplatin-induced neuropathy by 4-MC accelerates bone marrow (BM) regeneration, Example 8 showed that glial cell-derived neurotrophic factor fused to Fc (GDNF-Fc) acts specifically on SNS fibers to improve hematopoietic regeneration, and Example 9 confirms that anti-neuropathic agents are effective in hematopoietic regeneration.

### Example 1

The materials and methods used in the studies described in the following examples are disclosed below.

Mice.

Six- to sevenweek-old female C57BL/6J mice were purchased from National Cancer Institute (Frederick Cancer Research Center, Frederick, Md.). Adrb2 tm1Bkk/J mice were a gift from Dr. Gerard Karsenty, and can be obtained by one of skill in the art. All mice were housed at the Center for Comparative Medicine and Surgery at Mount Sinai School of Medicine. Experimental procedures performed on the mice were approved by the Animal Care and Use Committee of Mount Sinai School of Medicine.

Cisplatin Treatment.

To assess the role of chemotherapy in bone marrow transplantation, mice were injected intraperitoneally (i.p.; 10 mg/kg) with cisplatin (Teva) at a concentration of 0.2 mg/mL once a week for 7 weeks. To protect from kidney damage, mice were simultaneously subcutaneously (s.c.) injected with 1 mL of saline solution. Four weeks after the last injection of cisplatin, mice were euthanized for analysis, transplanted (see below) or mobilized with granulocyte colony-stimulating factor (G-CSF, see below).

6-Hydroxydopamine (60HDA) Treatment.

To induce acute peripheral sympathectomy, mice received two i.p. injections of 60HDA (Sigma; 100 mg/kg on day 0; 250 mg/kg on day 2). Three days after the last injection of 60HDA mice were euthanized for analysis, transplanted (see below) or injected with 5-fluorouracil (see below).

Bone Marrow Transplantation.

Mice were irradiated (1,200 cGy, two split doses, 3 hours apart) in a Cesium Mark 1 irradiator (JL Shepperd & associates). Three hours later, the indicated number of BMNCs was injected retroorbitally in the irradiated recipients under isoflurane (Phoenix Pharmaceuticals) anesthesia. Mice were allowed to recover and analyzed at the indicated time points.

5-Fluorouracil (5FU) Treatment.

To induce bone marrow ablation and force quiescent HSC to proliferate, 5FU (250 mg/kg; Sigma) was injected i.v. under isoflurane (Phoenix Pharmaceuticals) anesthesia. Mice were allowed to recover and analyzed at the indicated time points.

Inhibition of β3 Adrenergic Receptors In Vivo.

To investigate the role of β2 or β3 adrenergic receptors in bone marrow regeneration, β3 adrenergic signaling was blocked in wild-type or Adrb2^{tm1Bkk/J} mice by injecting the β3-specific antagonist SR59230A (5 mg/kg, i.p.; Sigma), daily for 3 days.

Generation of GDNF-Fc.

The murine cDNA for glial cell-line derived neurotrophic factor (Gdnf) was obtained from Open Biosystems. This cDNA was amplified and restriction sites were added for cloning with the following primers Forward: ACG CTA GCA ATG GGA TTC GGG CCA CTT (SEQ ID NO:1); Reverse: CGA GAT CTG CGA TAC ATC CAC ACC GTT TAG (SEQ ID NO:2). The PCR product was purified and cloned into the PCL5.1neg plasmid to generate PCL5.1neg-GDNF. This plasmid was purified and transfected into 293T cells. Two days after transfection, the supernatant was collected and GDNF-Fc purified in a Protein G-sepharose column (Pierce). To assess functional activity of GDNF-Fc recombinant protein, PC12ES cells were cultured in DMEM supplemented with 5% FBS, 10% horse serum, sodium-pyruvate (Gibco), L-Glutamine (Gibco) and penicillin/streptomycin (Gibco) for 3 days and then the media was replaced with DMEM supplemented with 1% horse serum, sodium-pyruvate (Gibco), L-Glutamine (Gibco), penicillin/streptomycin (Gibco) and varying amounts of GDNF-Fc to induce differentiation. Seven days later the percentage of PC12 ES cells with two or more dendrites was scored under an inverted microscope. For each concentration of GDNF-Fc, 10 fields were analyzed.

Neuroprotection with 4-Methylcatechol (4-MC) or GDNF-Fc.

To induce neuroprotection from cisplatin, mice were injected intraperitoneally with 4-MC (10 μg/kg; Sigma) daily for the 7 weeks of cisplatin treatment. Neuroprotection was also induced in cisplatin-treated mice with daily subcutaneous injections of recombinant GDNF-Fc (5 μg per mouse) during 2 weeks immediately after the last injection of cisplatin. To induce neuroprotection from 60HDA, mice were injected with 4-MC (10 μg/kg; i.p.) or GDNF-Fc (5 μg per mice; s.c.) for 5 days, starting the treatment the same day as the first injection of 60HDA.

G-CSF-Induced Mobilization.

Mice received G-CSF (250 mg/kg/day) s.c. every 12 hours for 5 days. Due to circadian oscillations on HSC mobilization, the last dose of G-CSF was administered 1 hour before blood collection at Zeigeber time 5.

Blood and Bone Marrow Analyses.

Blood was harvested by retro-orbital sampling of mice anesthetized with isoflurane and collected in polypropylene tubes containing ethylenediaminetetraacetic acid (EDTA). Blood parameters were determined with an AcT differential counter (Beckman-Coulter). CFU-C assays were performed as described in He et al., Ann. Rev. Cell. Dev. Biol. 25:377 (2009), incorporated herein by reference. For flow cytometry, red blood cells were lysed thrice for 5 minutes at 4° C. in 0.15 M NH₄Cl, cells were washed once in ice-cold PBS and counted in a hemocytometer. To determine Lin⁻Sca1⁺c-kit⁺ or Lin⁻Sca1⁺c-kit⁺ft13⁻ numbers, 10⁶ cells were stained with the Mouse Lineage Panel (BD Biosciences) together with FITC-conjugated anti-Sca-1 antibody (BD Biosciences), PE-Cy7-conjugated anti-c-kit antibody (eBioscience) and PE-conjugated anti-flt3 antibody (eBioscience). Cells were further stained with streptavidin-Cy5 (Jackson Immunotech) and analyzed with a BD LSR11 system (BD Biosciences). Bone marrow was harvested by flushing the bone with 1 mL of ice-cold PBS, red blood cells were lysed once for 5 minutes at 4° C. in 0.15 M NH₄Cl, cells were washed once in ice-cold PBS and counted with a hemocytometer. CFU-C and Lin⁻Sca1⁺c-kit⁺ft13⁻ numbers were determined as above. In some experiments, CD150⁺CD48⁻ cell numbers were determined by staining 5×10⁶ cells with PE-anti-CD48 antibody (BD Biosciences) and PE-Cy7-anti-CD150 antibody (Biolegend).

Cell Cycle Analysis of Lin⁻Sca1⁺c-Kit⁺ Cells.

Forty-eight and twenty-four hours before analysis, saline- or 60HDA-treated mice received i.p. injections of BrdU (100 μg; BD Biosciences). On day 0, mice were euthanized and BMNC purified and stained as indicated above. Cell cycle was determined by staining for BrdU-labeled cells with the APC BrdU Flow Kit (BD Biosciences) following manufacturer's instructions. Cells were then analyzed in a BD LSR11 system (BD Biosciences).

Quantification of Sensory Neuropathy by the Heated Pad Assay.

To evaluate the effect of different treatments on the sensory response, the hot-plate test was performed as described in Raaijmakers et al., Curr. Opin. Hematol. 15:301 (2008), incorporated herein by reference. An Isotemp Dryblock (Fisher Scientific) was heated to, and maintained at, 50° C. Mice were individually placed on top of the heated surface and the time to the first episode of nociception (jumping or paw licking) was measured. The cut-off time was 60 seconds. Between measurements, the heated surface was thoroughly cleaned with detergent and ethanol and the temperature was allowed to stabilize at 50° C.

Immunofluorescence Analyses.

Bones were collected and fixed for 1 hour in 4% paraformaldehyde (PFA) in PBS (Electron Microscopy Sciences) at 4° C. They were then post-fixed overnight in 1% PFA in PBS at 4° C. and cryoprotected for 24 hours in 30% sucrose. Bones were then included in OCT (Tissue Tek), sectioned (14 μm sections) in a Cryostat, and mounted on CFSA 4× Slides (Leica). TH⁺ immunofluorescence staining was performed as previously described in Mendez-Ferrer, et al., Nature 452:442 (2008), incorporated herein by reference. For each mouse analyzed, the number of nerve fibers in 6 fields was quantified and plotted as per mm². For whole-mount immunofluorescence, calvaria were harvested by cutting along the temporal lines of the skull and immediately fixed in methanol. Bone tissues were blocked/permeabilized in PBS containing 20% FCS and 0.5% Triton and stained with APC-conjugated anti-PECAM CD31 antibody and TH (Millipore). Signal amplification for TH staining was achieved by using a signal amplification kit (Perkin Elmer). Whole-mount tissues were imaged face-down on an upright Olympus BX61WI microscope. The area between the frontal and parietal bones was identified by moving along the coronal suture and images were obtained from the same area for all mice, along the coronal vein on either side of the central vein. The numbers of individual nerve fibers running alongside blood vessels were quantified and plotted as per 100 μm vessel segment. All images were processed using Slidebook software (Intelligent Imaging Innovations, Inc.).

Statististical Analyses.

All data are represented as mean±standard error of the mean. Comparisons between two samples were done using the Student's t test. Multivariate analyses were performed using one-way ANOVA and Tukey post analysis test. Log Rank analyses were used for Kaplan-Meier survival curves. *p<0.05; **p<0.01; ***p<0.001; ns: non-significant.

### Example 2

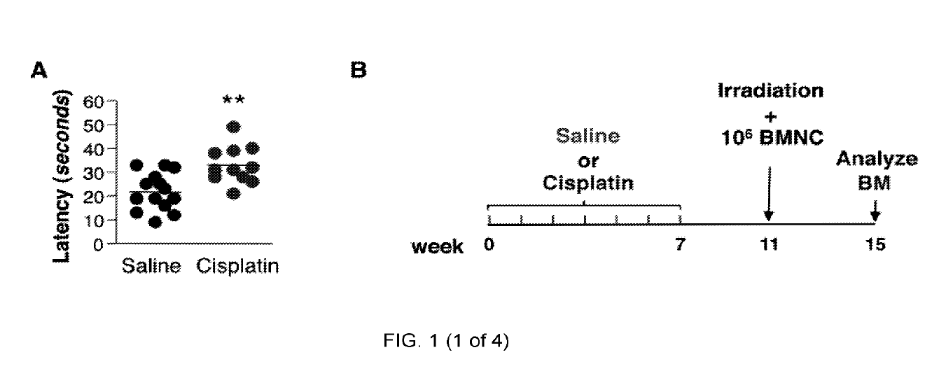

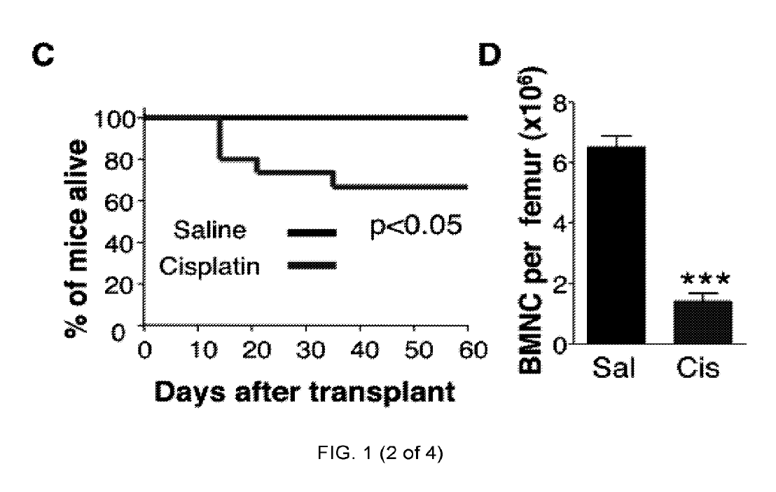

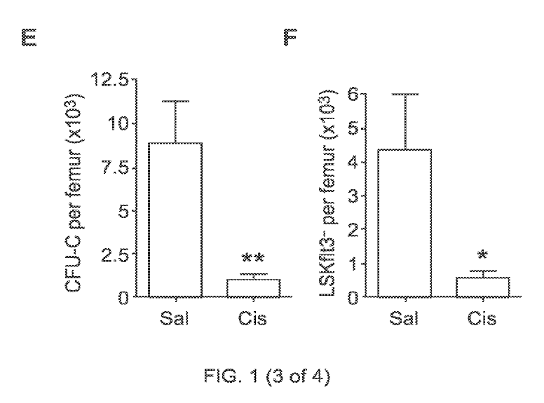

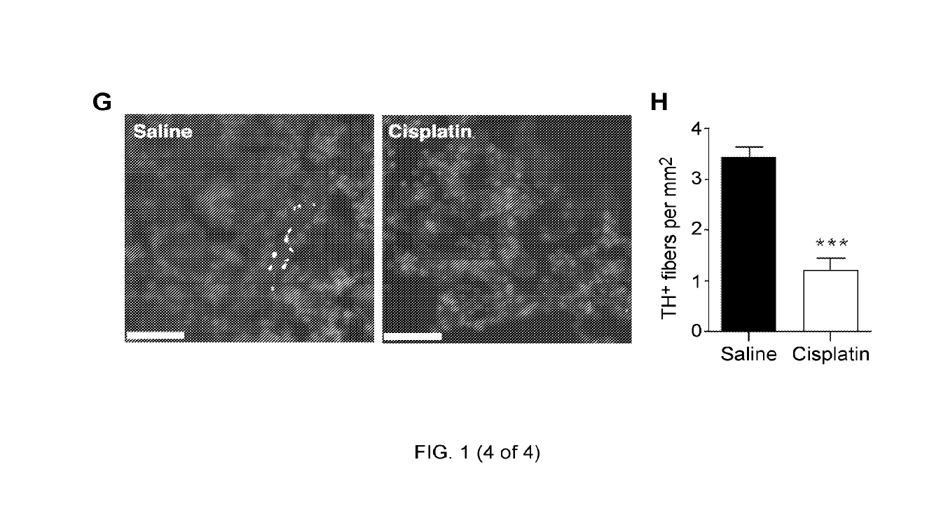

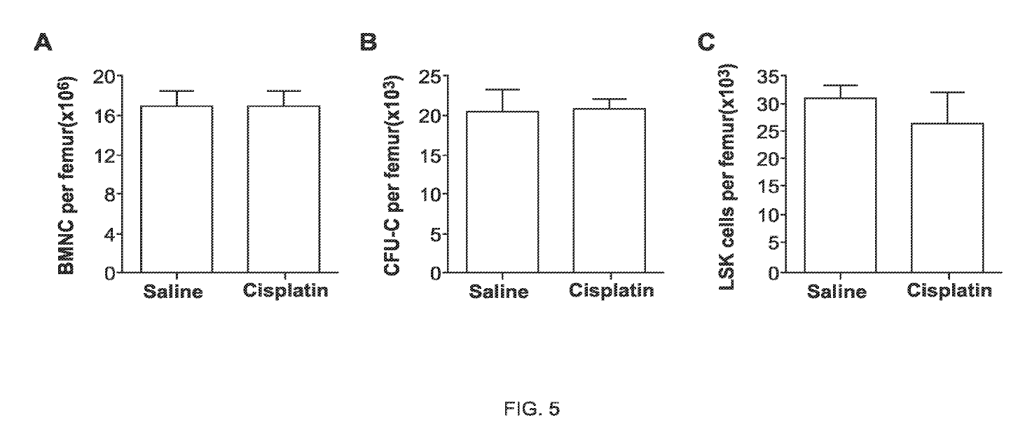

Mice were treated with seven weekly injections of cisplatin, a protocol that reproducibly induce sensory neuropathy similar to that seen clinically. One month later, hematopoiesis had completely recovered as measured by bone marrow cell, progenitor cell (CFU-C) and Lin⁻Sca1⁺c-kit⁺ cell counts (FIG. 5A-C). Mice continued to exhibit a sensory neuropathy at this time, however, as determined by increased latency time in a nociception assay (FIG. 1A). When cisplatin- or vehicle-treated mice were lethally irradiated and transplanted with fresh bone marrow nucleated cells (BMNC) from healthy donors (FIG. 1B), long-term survival in the cisplatin group was significantly reduced (by 33%; FIG. 1C). Increased lethality was due to reduced hematopoietic activity because the bone marrow of surviving cisplatin-treated mice was aplastic (FIGS. 1D-F), and showed dramatic reductions in the number of progenitors (FIG. 1E) and Lin⁻Sca1⁺c-kit⁺flt3⁻ HSCs (FIG. 1F). These data indicate that cisplatin treatment alters the host bone marrow microenvironment, impairing hematopoietic recovery after transplantation of healthy hematopoietic stem and progenitor cells.

### Example 3

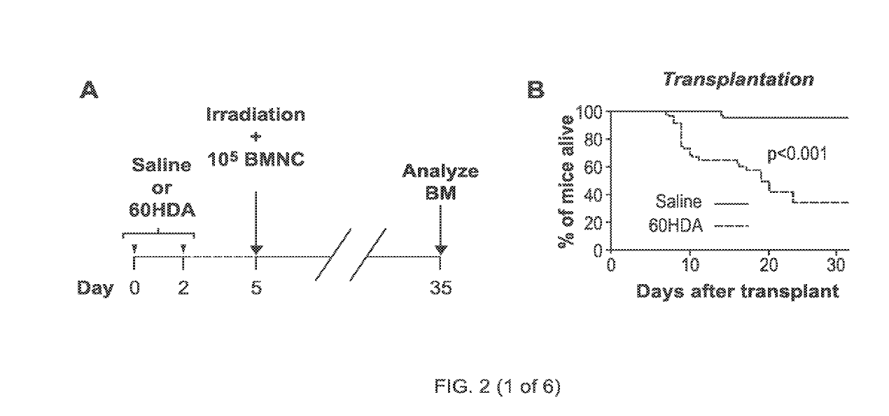

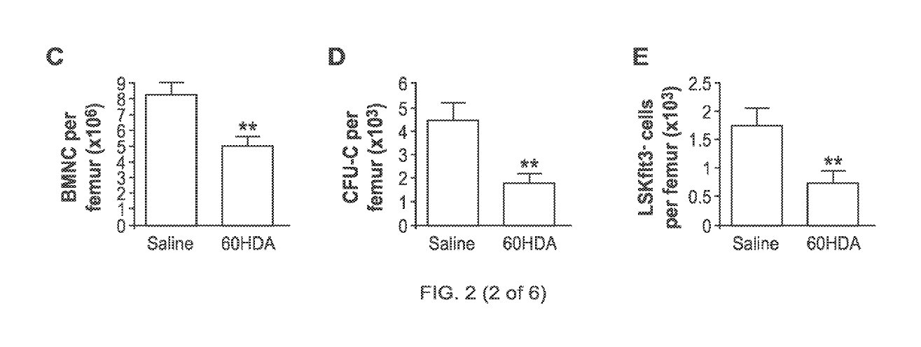

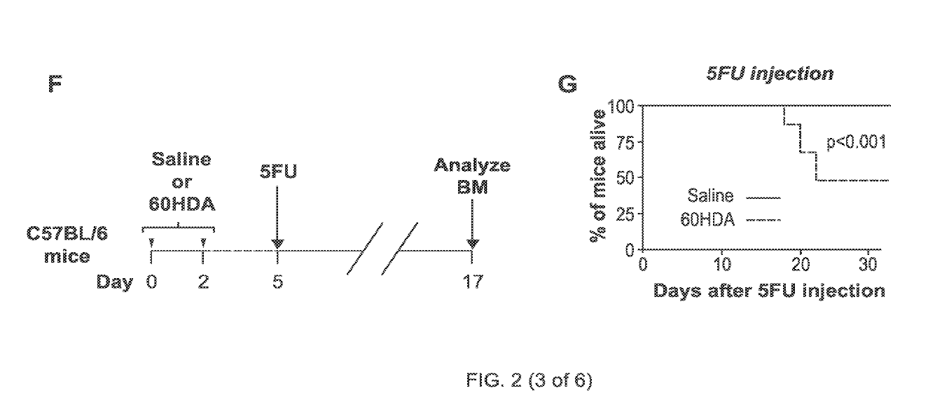

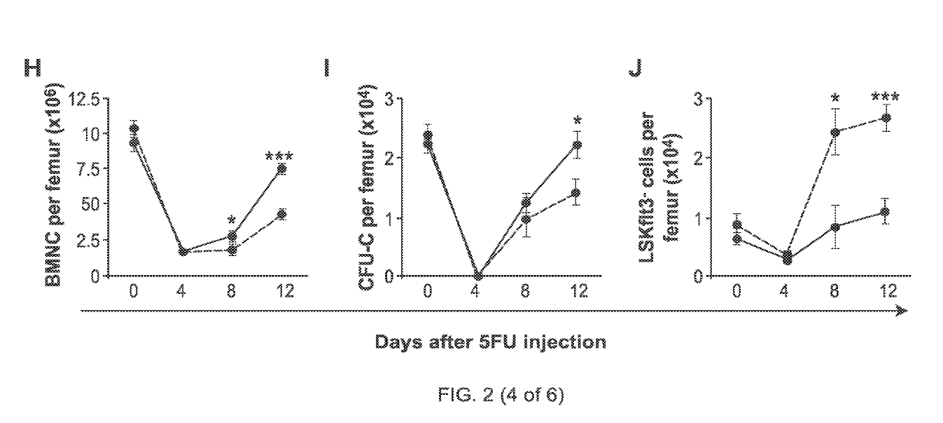

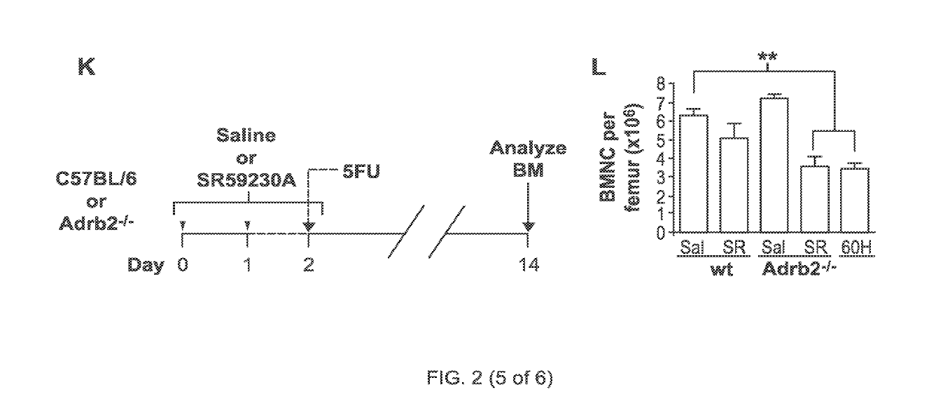

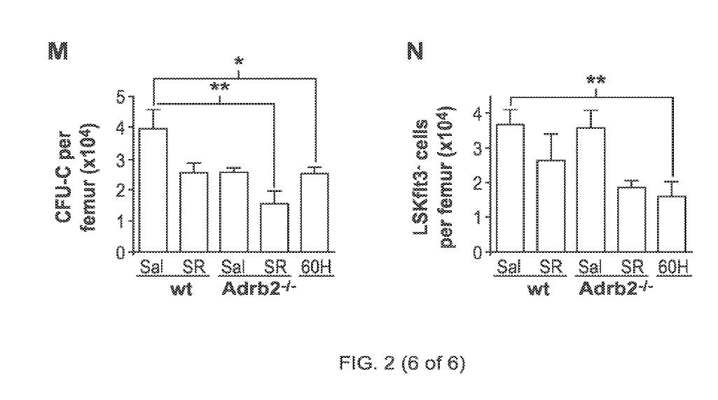

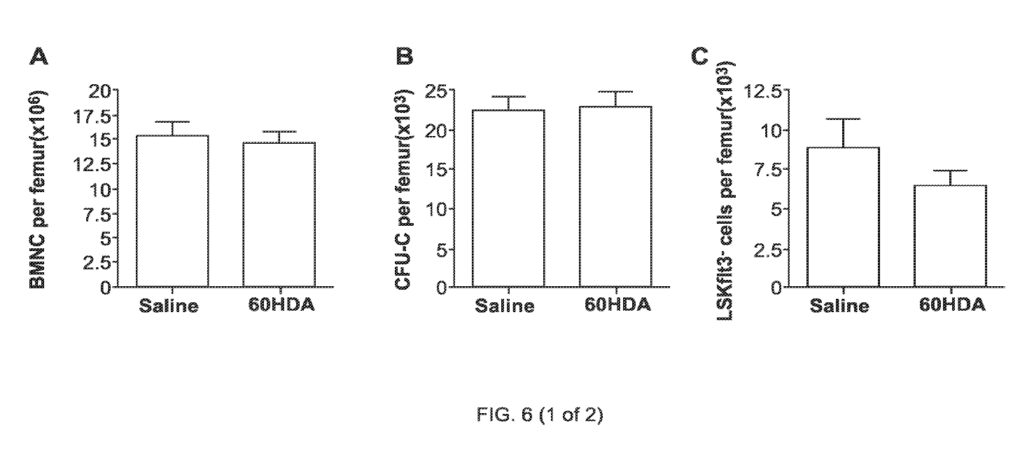

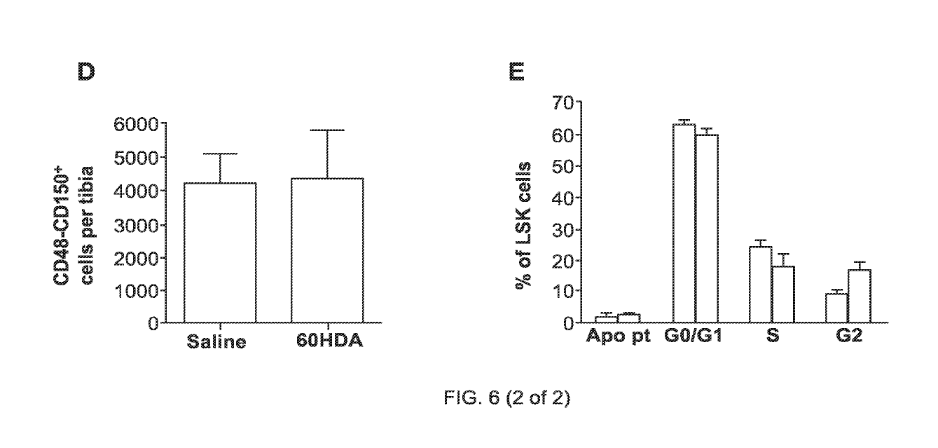

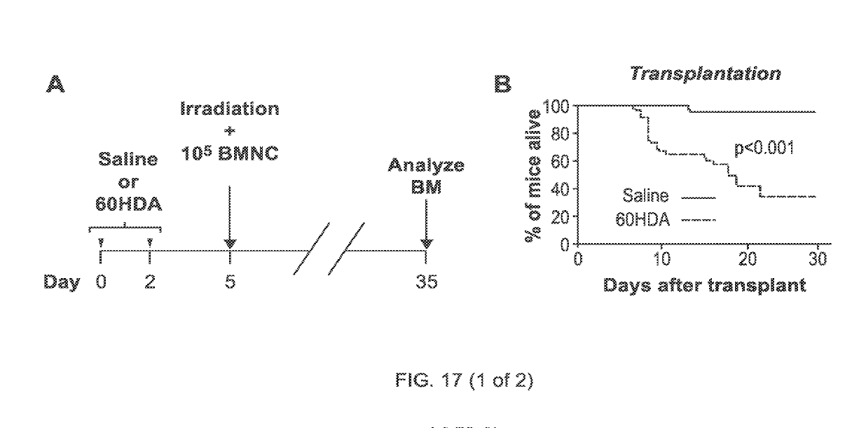

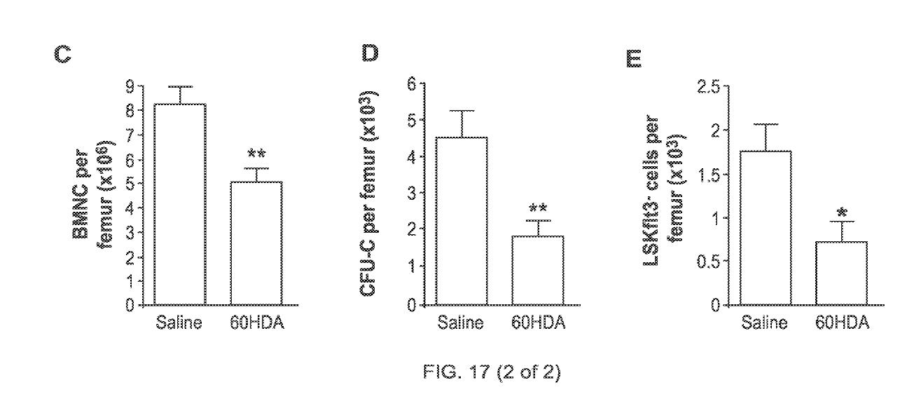

Cisplatin-induced neuropathy has been reported to affect largely sensory nerves. To assess whether cisplatin also caused a sympathetic neuropathy in the bone marrow, bone marrow SNS fibers were stained with an antibody against the catecholaminergic enzyme tyrosine hydroxylase (TH). Cisplatin treatment reduced the density of TH+fibers by 65% compared with vehicle control (FIG. 1G-H). To evaluate more specifically whether sympathetic innervation was required for hematopoietic regeneration after transplantation, the SNS was denervated by treatment with 6-hydroxydopamine (60HDA). Consistent with work in the art, 60HDA treatment by itself did not alter BM cellularity, CFU-C, Lin⁻Sca1⁺c-kit⁺flt3⁻, CD48⁻CD150⁺ cell numbers or Lin⁻Sca1⁺c-kit⁺ cell cycling (FIG. 6A-E). Transplantation of wild-type BMNC in lethally irradiated 60HDA- or saline-treated mice (FIGS. 2A and 17A), however, led to a significant increase in mortality within the 60HDA group (FIG. 2B, p<0.001; Logrank test; FIG. 17B). BM analysis 30 days post-transplantation revealed significant reductions in hematopoietic recovery in 60HDA-treated transplanted mice compared to saline controls (FIG. 2C-E; FIG. 17C-E). These experiments demonstrate that SNS denervation reduces HSC engraftment after transplantation, supporting a role for cisplatin-induced neuropathy in impeding or preventing hematopoietic regeneration.

Further and in contrast to the failure of 60HDA to affect BM cellularity, CFU-C, LSKF, CD48⁻CD150⁺ cell numbers, or Lin⁻Sca1⁺c-kit⁺ cell cycling, transplantation of wild-type BMNC in lethally irradiated 60HDA- or saline-treated mice (FIG. 17A) led to a significant increase in mortality within the 60HDA group (FIG. 17B) and delayed hematopoietic recovery 30 days post-transplantation (FIG. 17C-E). Thus, SNS denervation reduces HSC engraftment after transplantation, further establishing that chemotherapy-induced neuropathy prevents or inhibits hematopoietic regeneration.

### Example 4

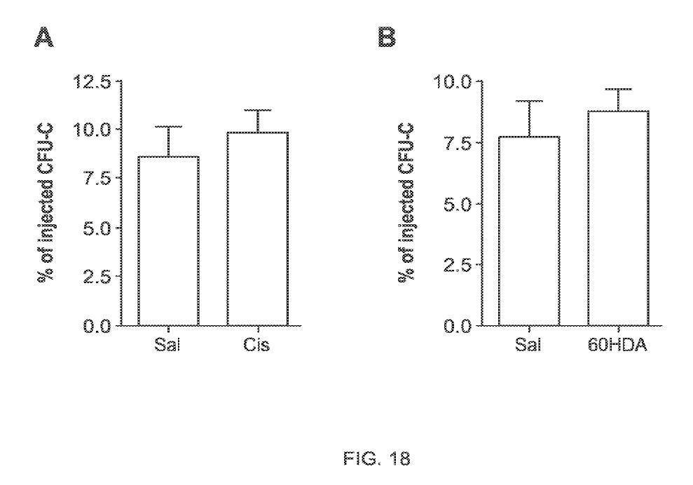

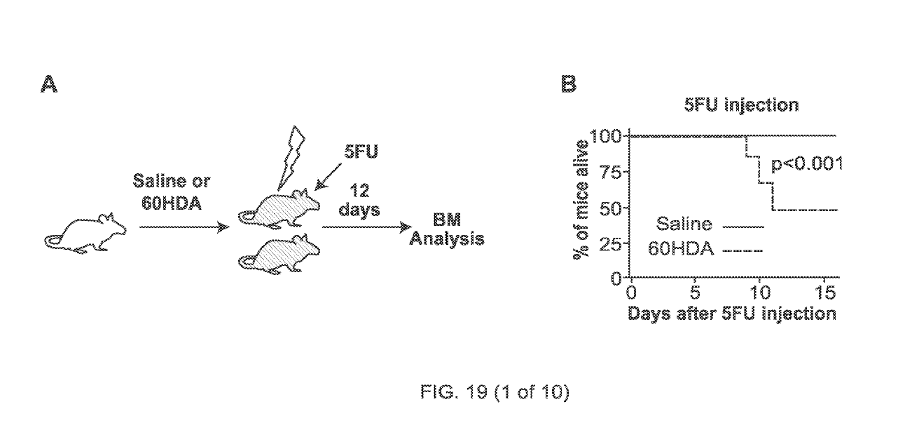

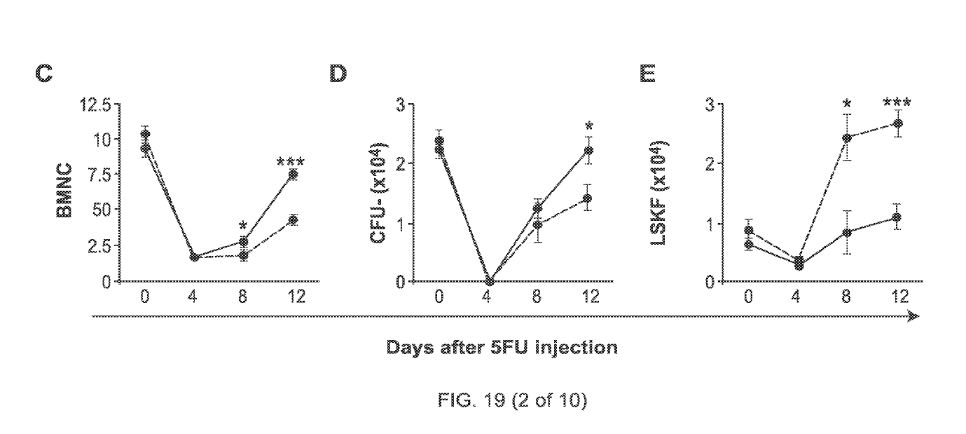

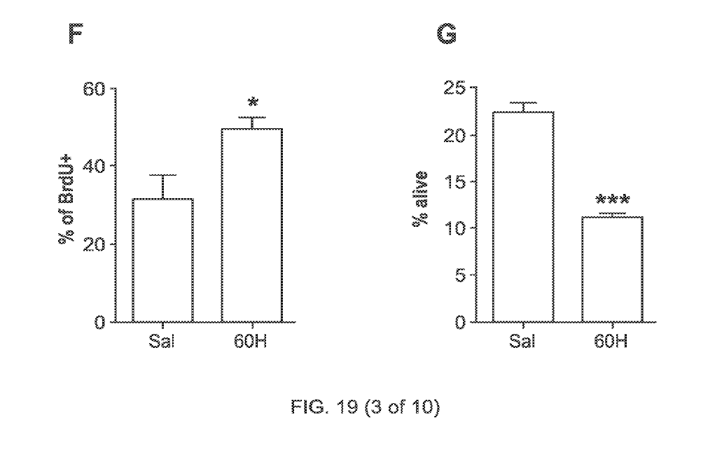

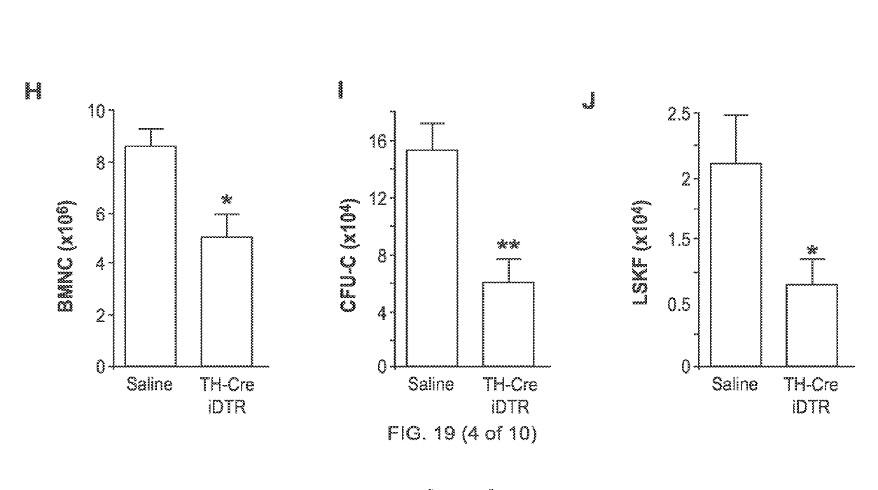

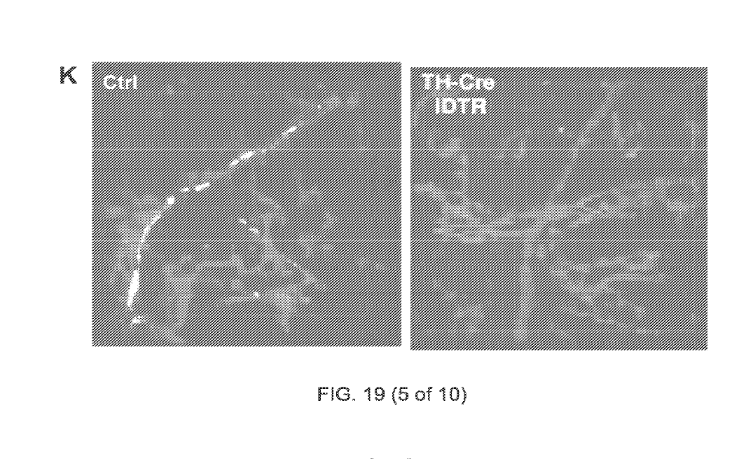

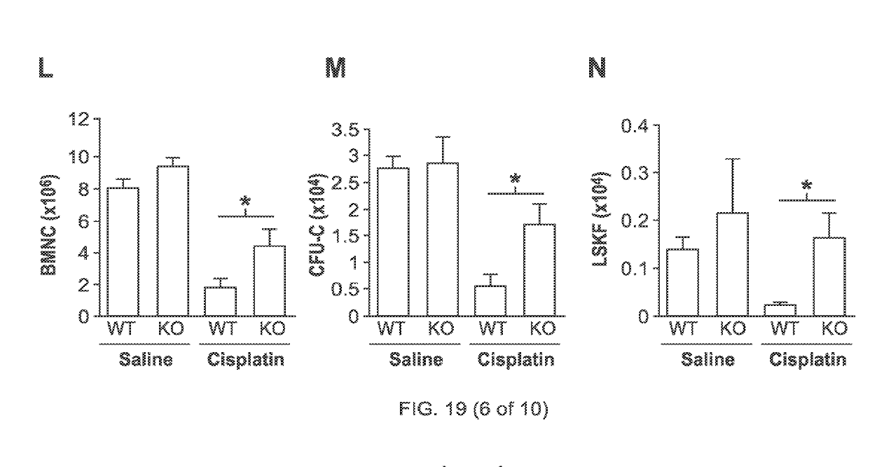

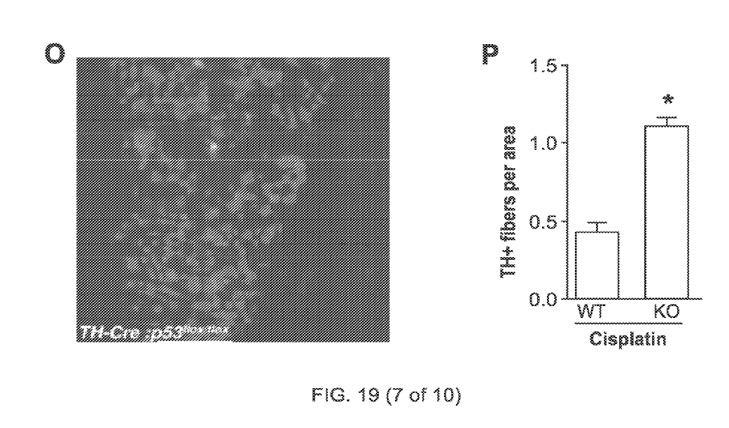

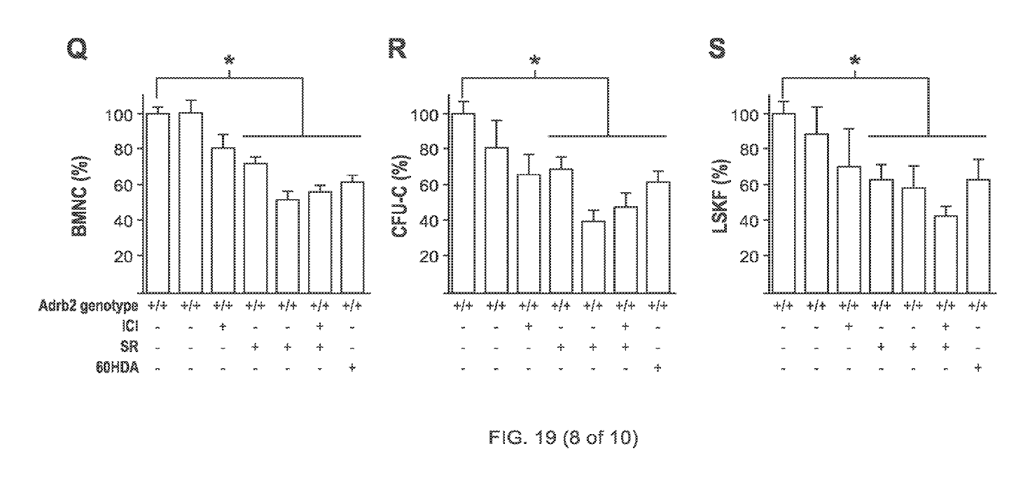

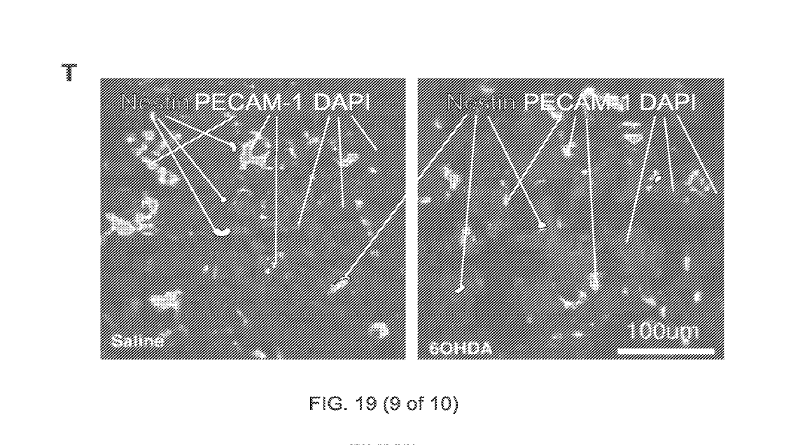

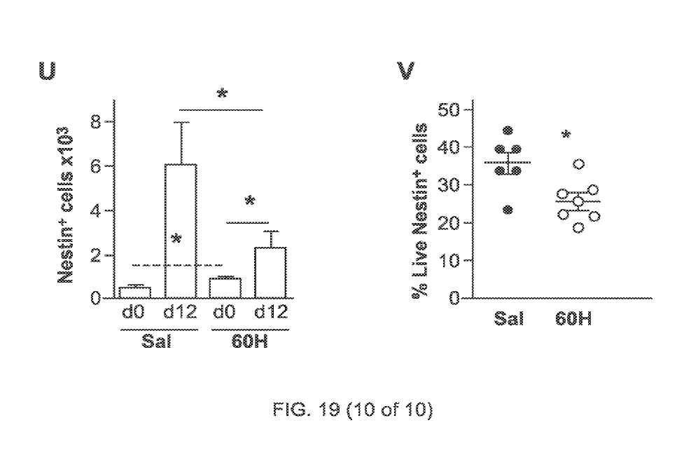

Transplantation of HSCs is a complex process that requires homing to the bone marrow and migration to the appropriate niche for survival and proliferation. No differences were found in CFU-C homing efficiency in 60HDA- or cisplatin-treated mice when compared to control mice (FIG. 18A-B). To further confirm that reduced BM recovery was independent of homing, the response of saline-treated animals was tested following the administration of 5-fluorouracil (5FU), which ablates proliferating cells while inducing quiescent HSCs to repopulate the bone marrow in situ (FIGS. 2F and 19A). 5FU administration to 60HDA-sympathectomized mice dramatically reduced their survival (FIGS. 2G and 19B) due to BM failure (Table 3) and significantly impaired hematopoietic recovery at day 12 (FIGS. 2H-J and 19C-E).

TABLE 3
Table 3. Complete blood counts (CBC) of moribund 6OHOA-treated mice
at the indicated time points after injection of 5FU (250 mg/kg)

| Days after | WBC | RBC | Hgb | HCT | MCV | MCH | MCHC | CHCM | CH | RDW | HDW | PLT | MPV |
|---|---|---|---|---|---|---|---|---|---|---|---|---|---|
| 5FU | ×10 [symbol] ml | ×10 [symbol] ml | (g dl) | [symbol] | 1L | pg | g dL | g dL | pg | [symbol] | g dL | ×10 [symbol] ml | 1L |
| 8 | 0.9 | 3.0 | 3 | 12 | 40.4 | 9.9 | 24.5 | 32.8 | 13.3 | 130 | 2.34 | 410 | 11.3 |
| 8 | 1.1 | 1.8 | 0 | 7 | 40.3 | 0 | 0 | 31.8 | 12.8 | 11.9 | 1.78 | 90 | 12.4 |
| 8 | 1.5 | 5.3 | 6 | 22 | 41.5 | 10.7 | 25.9 | 31.2 | 12.9 | 12.2 | 1.74 | 620 | 10.9 |
| 9 | 0.1 | 2.8 | 3 | 11 | 40.1 | 9.7 | 24.3 | 33.7 | 13.5 | 12.5 | 2.27 | 30 | 11.4 |
| 9 | 0.2 | 2.8 | 3 | 11 | 40.8 | 9.1 | 22.3 | 33 | 13.5 | 12.6 | 2.34 | 20 | 15.7 |
| 10 | 0.3 | 3 | 4 | 13 | 43.7 | 12.8 | 29.3 | 29.3 | 12.8 | 12.2 | 1.67 | 250 | 19 |
| 10 | 0.6 | 2.5 | 6 | 10 | 42.5 | 18.9 | 44.4 | 30.5 | 12.9 | 13.2 | 2.03 | 450 | 16.2 |
| 11 | 1.1 | 3.2 | 3 | 14 | 43 | 10.5 | 24.3 | 30.7 | 13.2 | 11.3 | 1.95 | 140 | 15.4 |
| 12 | 3.4 | 3.6 | 3 | 15 | 40.5 | 9.3 | 22.9 | 31.1 | 12.6 | 13.5 | 2.31 | 2600 | 6.7 |

[symbol]

indicates data missing or illegible when filed

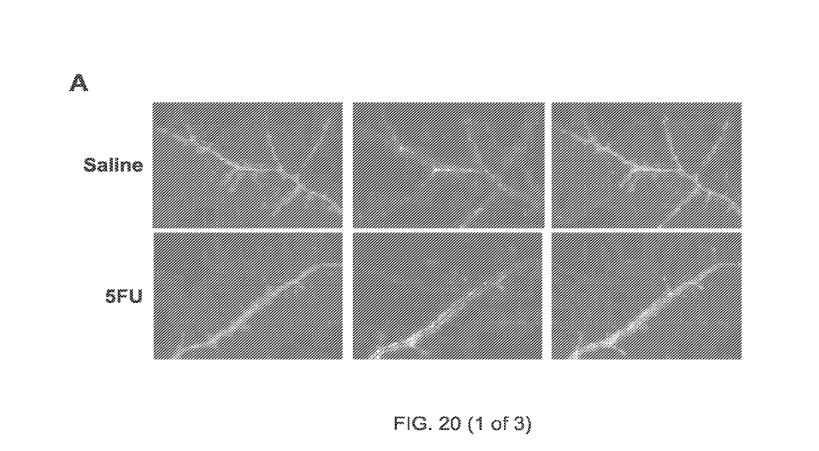

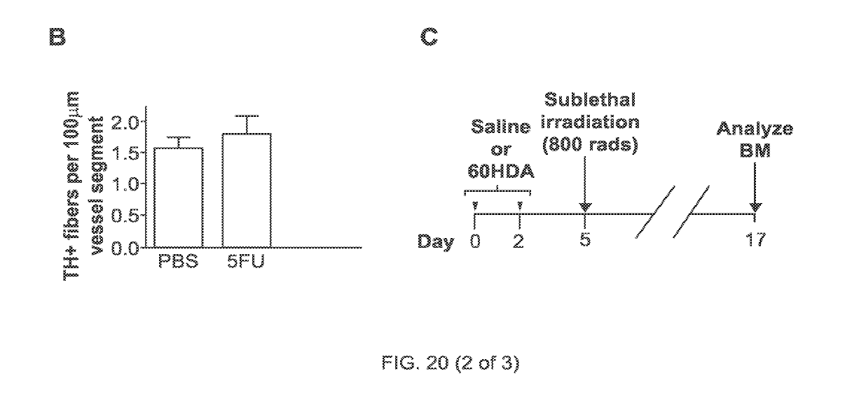

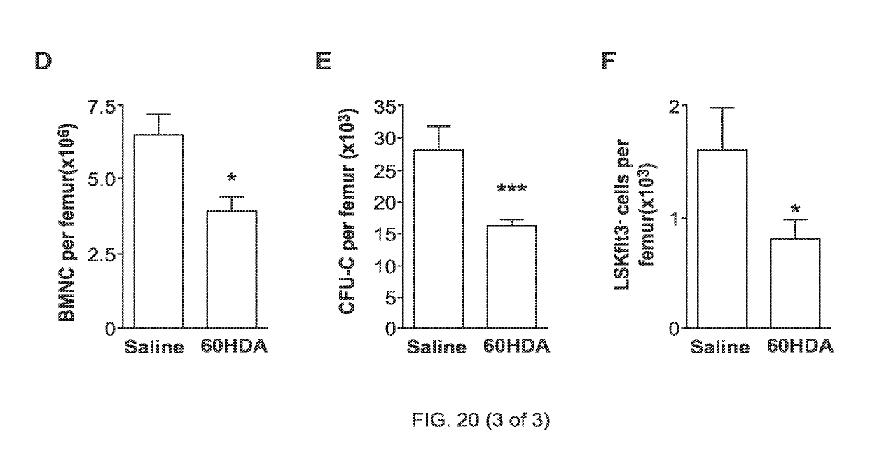

Analyses of the kinetics of BM regeneration revealed that the number of HSCs (Lin⁻Sca1⁺c-kit⁺flt3⁻ cells) increased very rapidly in saline-treated mice (6.5-fold) between days 4 and 8 followed by a more moderate expansion between days 8 and 12 (FIGS. 2J and 19E). In contrast, Lin⁻Sca1⁺c-kit⁺flt3⁻ cell expansion was blunted in 60HDA-treated mice (FIGS. 2J and 19E). Eight days after 5FU injection, LSK cells in 60HDA-treated mice showed increased proliferation (FIG. 19F) coupled to reduced viability (FIG. 19G). Similar, but delayed and more modest, differences were observed in the recovery of progenitors (FIG. 2I) and overall cellularity (FIG. 2H). These data indicate that while SNS signals are dispensable for steady-state HSC homeostasis, they are involved in maintaining the precise balance between HSC self-renewal and differentiation during BM regeneration, increased proliferation and increased apoptosis. Chronic 5-Fluorouracil treatment can induce neuropathy, but no nerve toxicity was detected after acute 5-FU injection in WT mice (FIG. 20A-B). Reduced BM recovery was also observed in 60HDA-treated mice after sublethal irradiation (FIG. 20C-F). These results indicate that reduced BM recovery in 60HDA mice is not due to a combination of 60HDA and 5FU SNS toxicity. To further confirm the role of the SNS in regeneration, TH-Cre mice (which express the Cre recombinase in catecholaminergic cells) were bred with iDTR mice (in which Cre recombination causes expression of the diphtheria toxin receptor (DTR)). Injection of diphtheria toxin to TH-Cre:iDTR mice followed by 5FU treatment led to reduced hematopoietic recovery (FIGS. 19H-J) and SNS ablation (FIG. 19K-L).

These experiments demonstrate that the SNS is required for BM recovery after genotoxic insult. Deletion of the p53 tumor suppressor in neurons increases their survival after genotoxic insult. To determine whether the SNS-injury observed in cisplatin-treated mice was responsible for reduced BM recovery, p53 was specifically deleted in catecholaminergic cells by breeding TH-Cre mice with p53^{flox/flox} mice to generate TH-Cre:p53^{flox/flox} mice. Control or TH-Cre:p53^{flox/flox} were then treated with cisplatin and BMT was performed, as described above. One month after BMT, cisplatin-treated TH-Cre:p53^{flox/flox} mice showed a strong increase in BM recovery when compared with WT-cisplatin-treated mice (FIGS. 19L-N) and a similar increase in the number of TH⁺SNS fibers in the BM (FIGS. 19O-P). This finding demonstrates that chemotherapy-induced neuropathy prevents BM regeneration. Circadian physiological HSC release is largely controlled via the β3 adrenergic receptors expressed by niche cells, whereas both β2 and β3 adrenergic receptors participate in enforced HSC mobilization. To determine which receptor(s) was/were required for 5FU-induced BM regeneration, wild-type or Adrb2^{-/-} mice were injected with saline, ICI118551 (a specific b2 antagonist) or SR59230A (a specific (β3 antagonist). While functional disruption of the β2-receptor did not severely compromise hematopoietic recovery, β3-blockade was sufficient to impair hematopoietic regeneration (FIG. 19Q-S). The most severe impairment, however, was observed when both β2 and β3 receptors were disrupted (FIG. 19Q-S). Thus, adrenergic signals transmitted by both β2 and β3 adrenergic receptors contribute to hematopoietic regeneration.

### Example 5

Circadian physiological HSC release is largely controlled via the β3 adrenergic receptors expressed by niche cells, whereas both P2 and β3 adrenergic receptors participate in enforced HSC mobilization. To determine which receptor(s) was required for 5FU-induced BM regeneration, wild-type or Adrb2^{-/-} mice were injected with saline or SR59230A, a specific β3 antagonist (FIG. 2K). While functional disruption of single adrenergic receptors partially compromised hematopoietic recovery, severe impairment in hematopoietic regeneration was observed when both β2 and β3 receptors were disrupted (FIG. 2L-N). Thus, adrenergic signals transmitted by the β2 and β3 adrenergic receptors are required for hematopoietic regeneration.

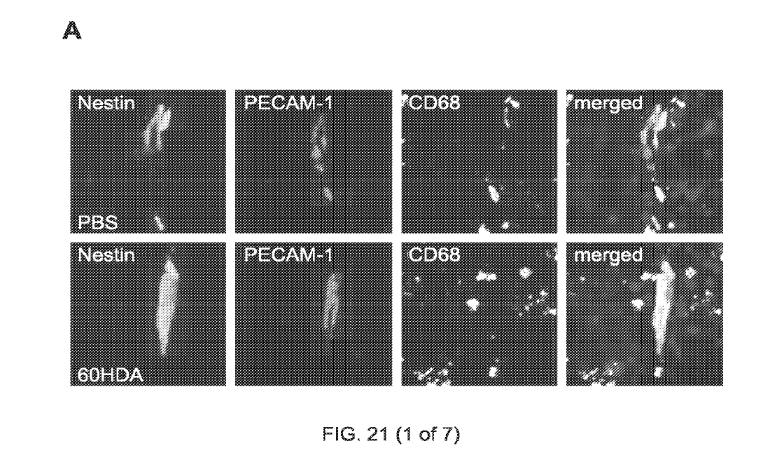

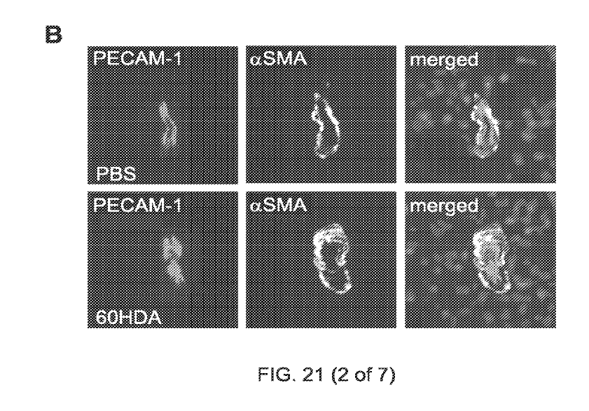

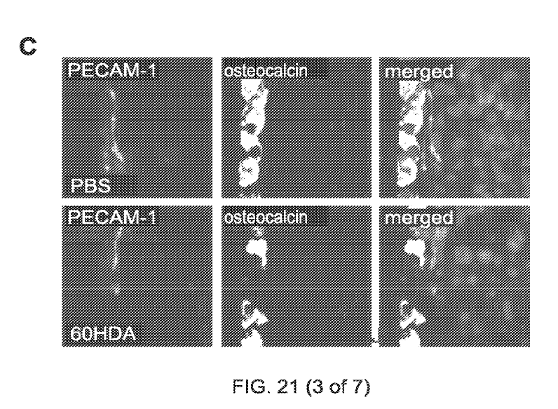

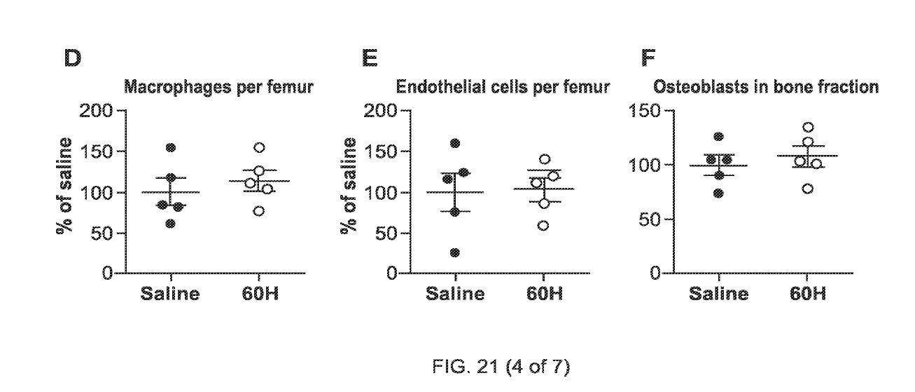

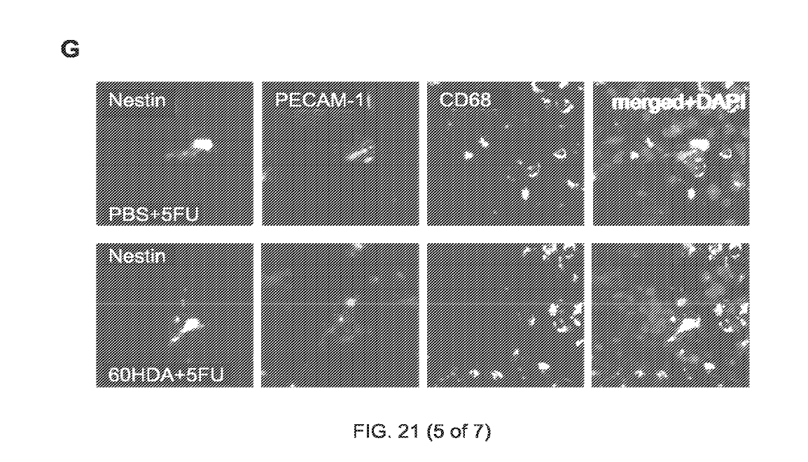

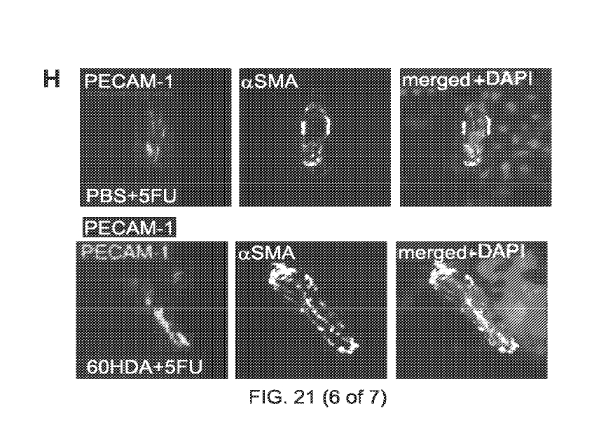

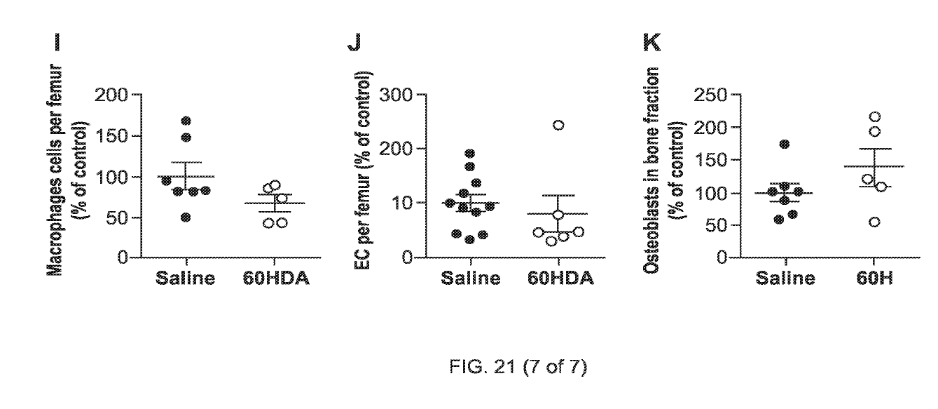

Since the SNS directs HSC trafficking by acting on Nestin⁺ niche cells through the β3 adrenergic receptor, the changes that occurred in the HSC niche after SNS injury and genotoxic insult were investigated. Prior 5FU administration immunofluorescence analyses did not reveal differences in the number of endothelial cells, osteoblasts, CD68⁺ cells (monocyte/macrophages) or α-SMA⁺ perivascular cells (FIG. 19T and FIG. 21A-C). Flow cytometry analyses also failed to detect differences in BM macrophages (which can regulate Nestin⁺ niche cells) and endothelial cells (FIG. 21D-E). A significant increase in the number of Nestin⁺ cells in 60HDA-treated mice (FIG. 19U) was detected, however. Osteoblast numbers were also slightly increased (FIG. 21F). Twelve days after 5FU injection, the number of Nestin⁺ cells in saline-treated mice had significantly increased (FIG. 19U), in agreement with earlier studies that showed osteoblastic cell expansion during BM recovery and indicating that the niche expands to accommodate increased proliferation of HSC. In contrast, in 60HDA-sympathectomized mice, this expansion is severely impaired and results in a severe deficit in Nestin⁺ cell numbers (FIG. 19U). Twelve days after 5FU injection, immunofluorescence and/or FACS analyses failed to detect differences in endothelial cells, BM monocyte/macrophages, α-SMA⁺perivascular cells between saline- and 60HDA-treated mice (FIG. 21G-K). Taken together, these results indicate a specific deficit in Nestin⁺ niche cells during BM recovery in sympathectomized mice. The reason for reduced Nestin⁺ numbers in SNS-injured mice during BM recovery was then investigated. Analyses of apoptosis revealed a significant increase in Nestin⁺ cell death in 60HDA mice 24 hours after 5Fu (FIG. 19V). These results indicate that one of the mechanisms through which the SNS acts on BM regeneration is by promoting survival and expansion/proliferation of Nestin⁺ cells after injury.

### Example 6

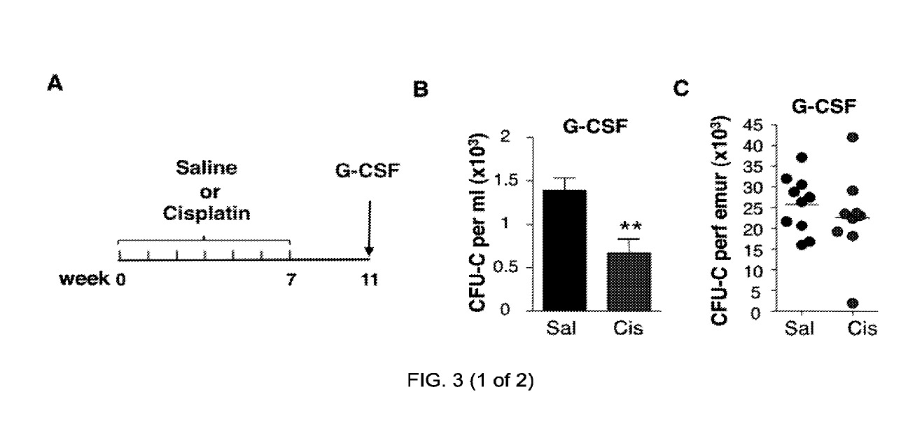

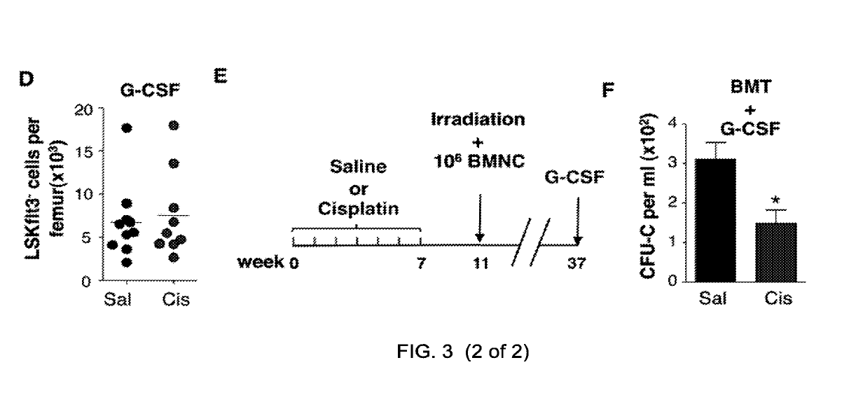

After acute administration of anti-cancer chemotherapy, hematopoietic recovery can be accompanied by a marked mobilization of HSC/progenitors in the bloodstream, revealing that the mobilization process may be associated with marrow regeneration. In addition, the G-CSF-induced mobilization takes several days to reach its peak, indicating the possible association between bone marrow remodeling and efficient mobilization. Therefore, the possibility that poor mobilization from prior chemotherapy treatment in cancer patients may be caused by bone marrow neuropathy was tested. To this end, mice were treated weekly with saline or cisplatin for 7 weeks, and G-CSF was administered to induce HSC/progenitor mobilization one month later (FIG. 3A). Remarkably, cisplatin-treated mice exhibited an approximately 50% reduction in the number of mobilized progenitors in the blood (FIG. 3B). Because no significant change in progenitors or Lin⁻Sca1⁺c-kit⁺ft13⁻HSC numbers was detected in the BM of these animals (FIG. 3C-D), the data show that the reduced mobilization was not due to lower numbers of HSC/progenitors from chemotherapy treatment. To completely rule out the possibility of a stem cell-autonomous defect, cisplatin and saline-treated mice were lethally irradiated and transplanted with fresh wild-type BMNC and allowed to recover for 16 weeks (FIG. 3E). At this time, BM recovery was complete and HSC content was identical in cisplatin- or saline-treated mice (FIG. 3C-D). In this setting, there was still an approximately 50% reduction in mobilization efficiency after G-CSF administration in cisplatin-treated mice compared to saline control (FIG. 3F), indicating that cisplatin treatment produced bone marrow neuropathy that markedly compromised HSC/progenitor trafficking.

### Example 7

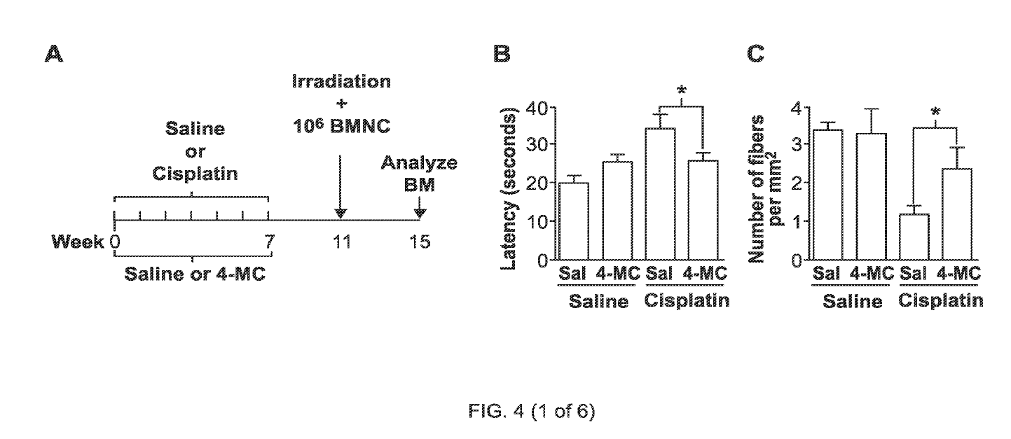

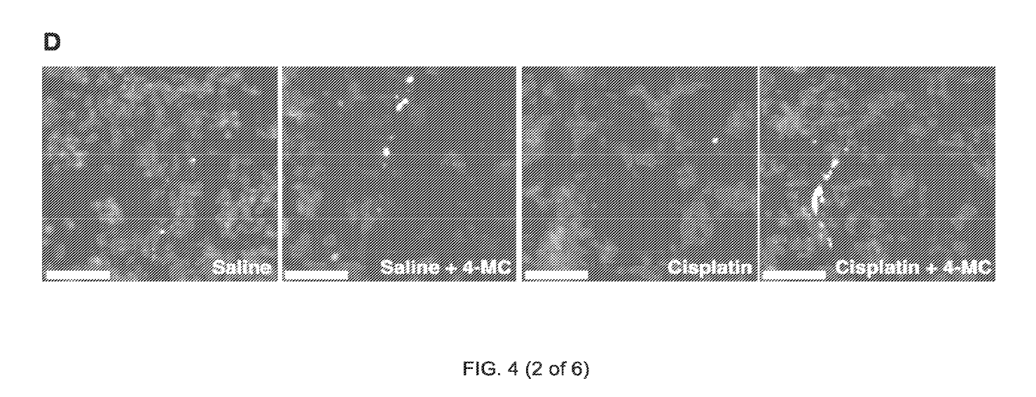

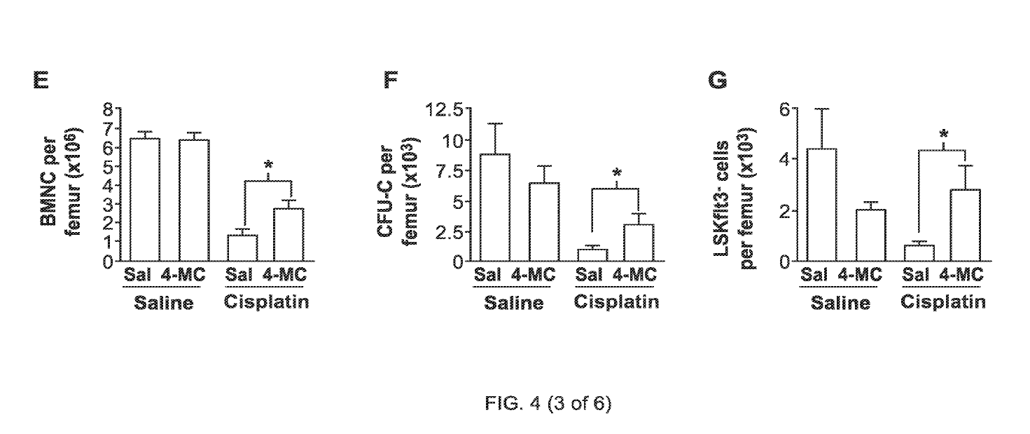

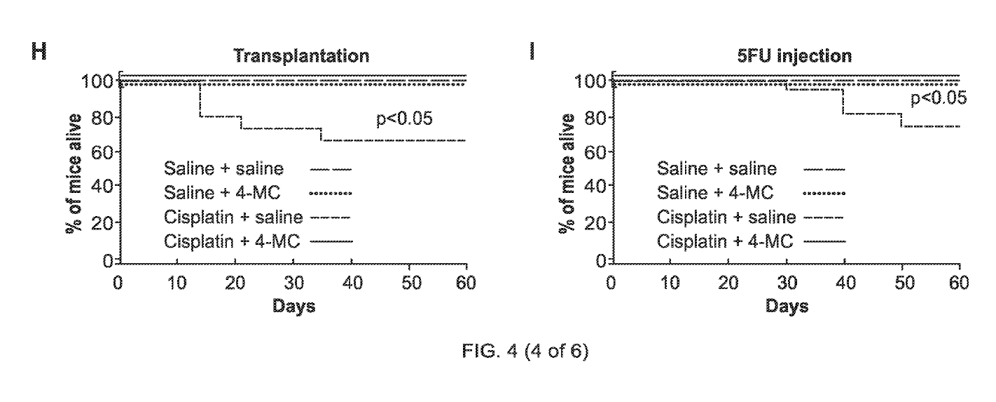

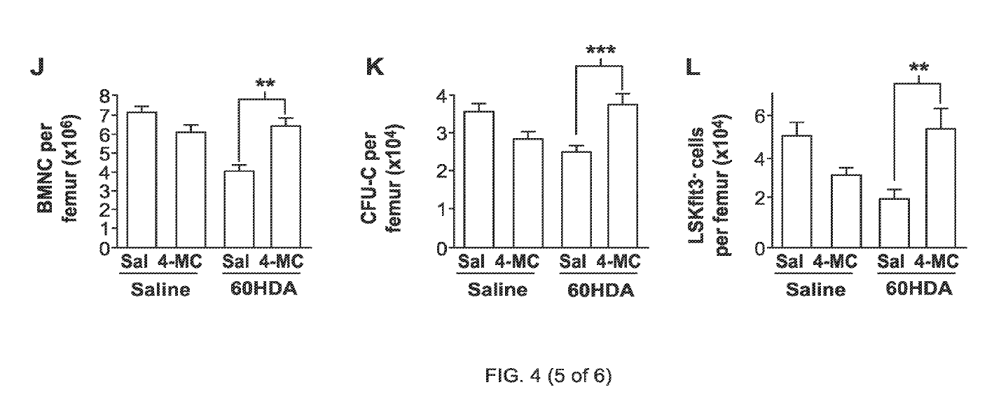

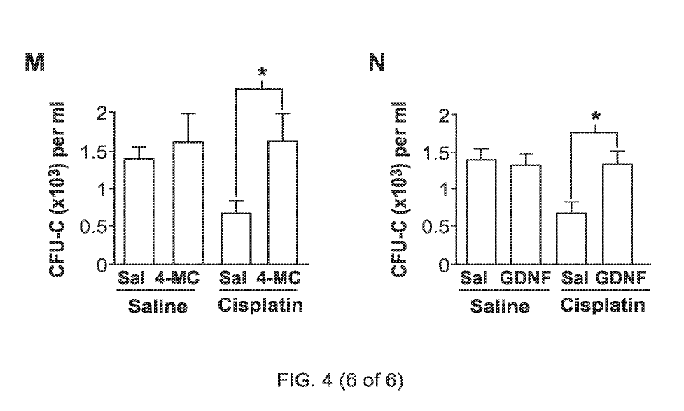

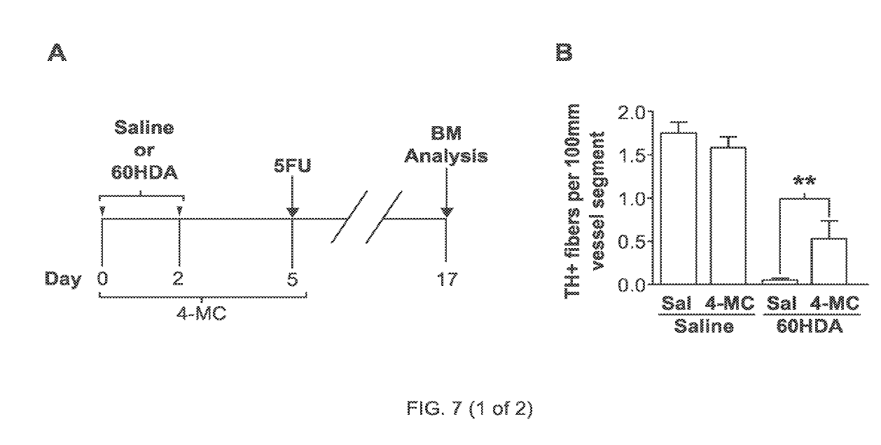

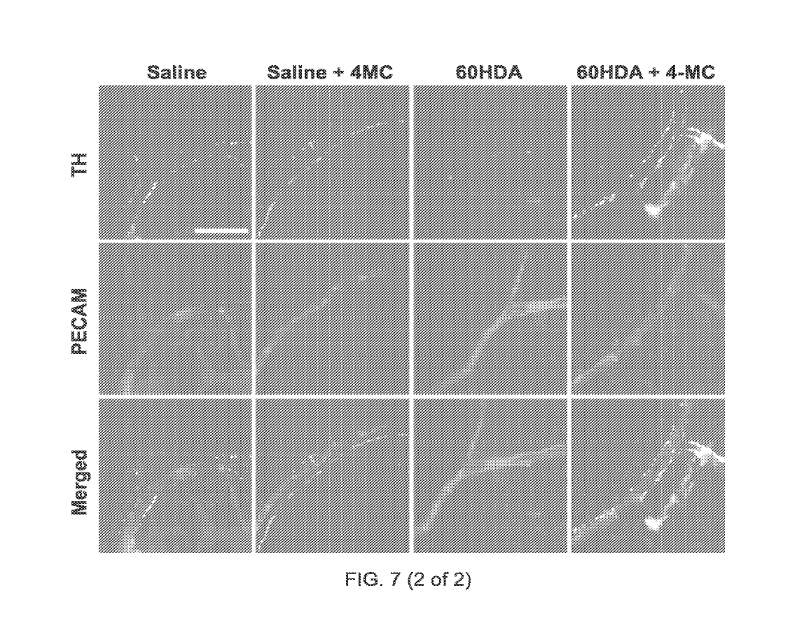

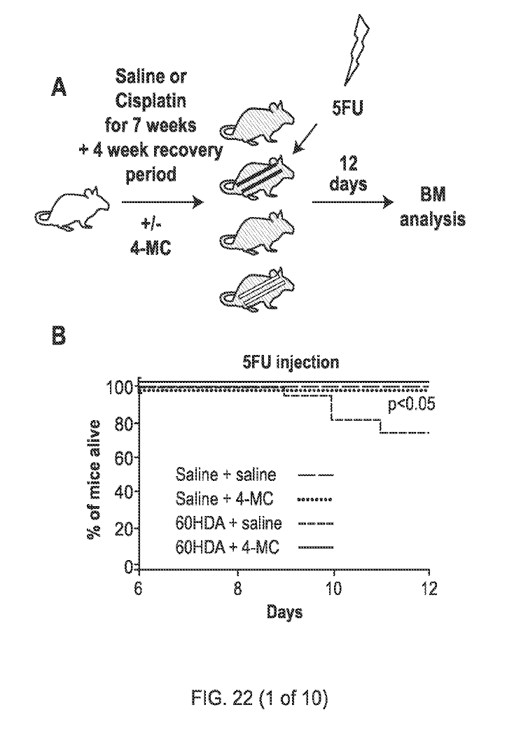

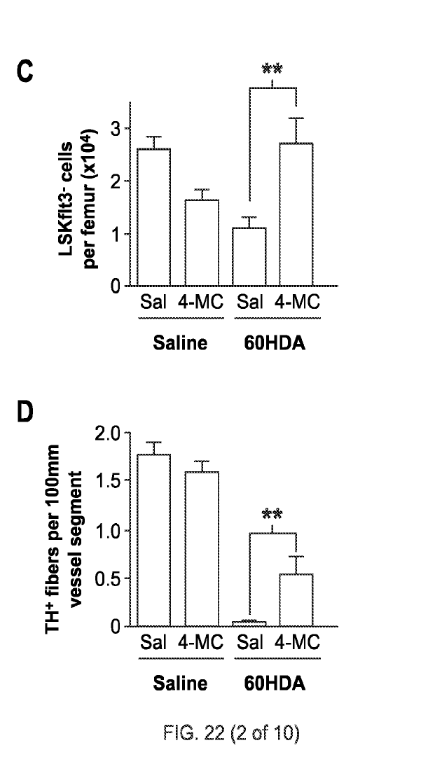

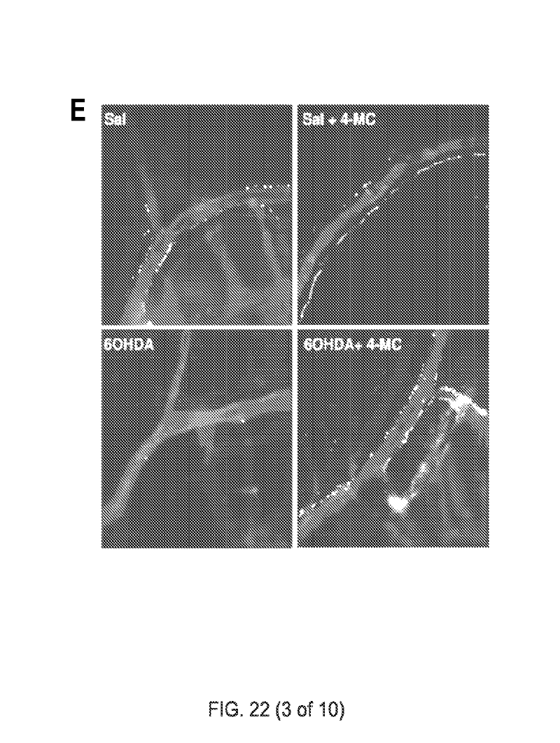

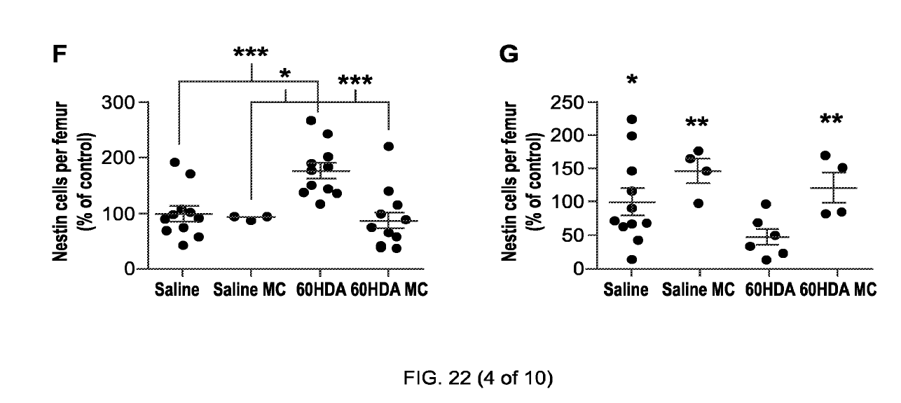

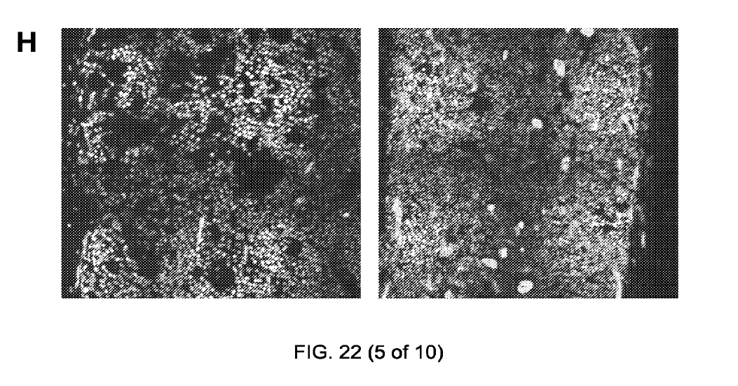

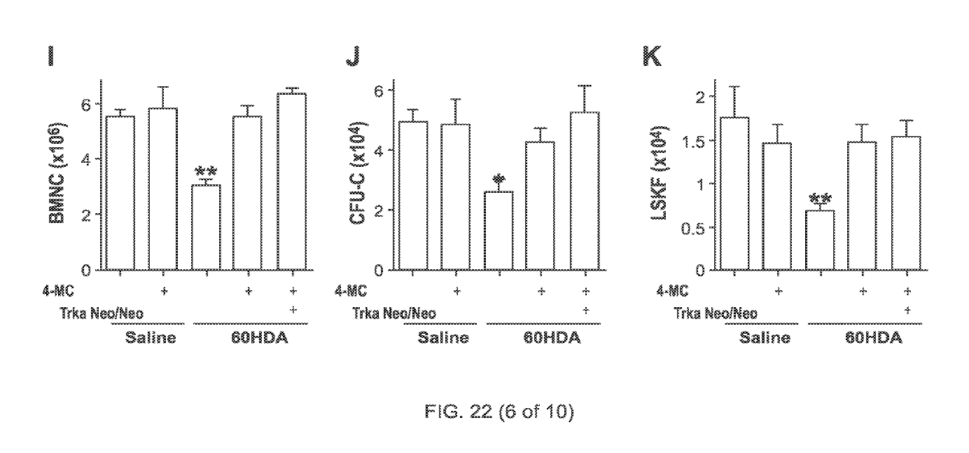

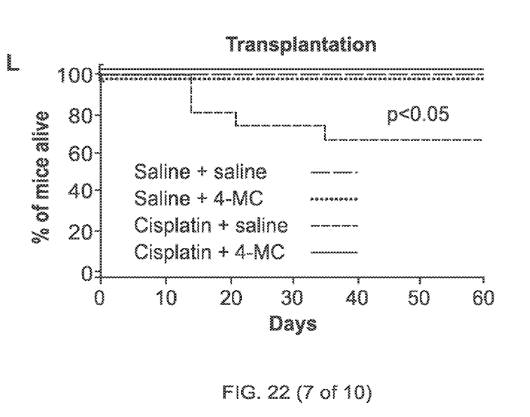

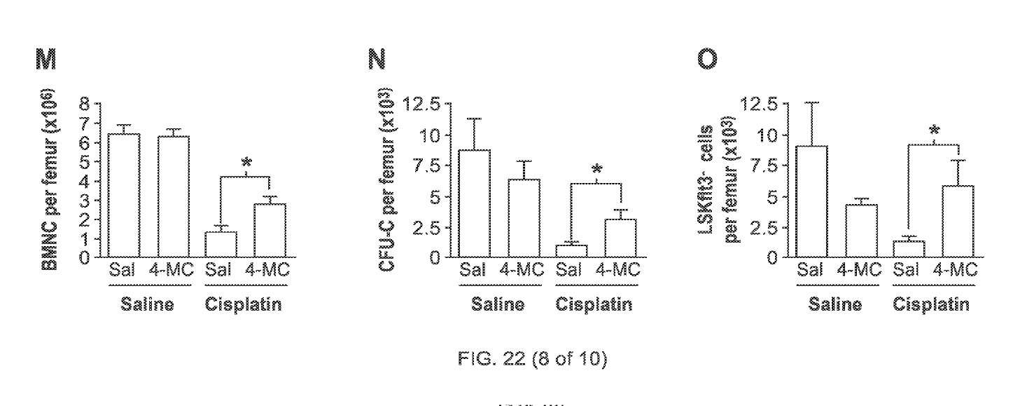

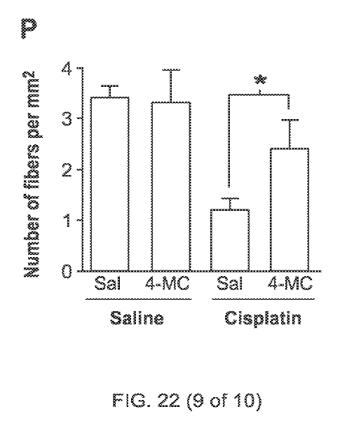

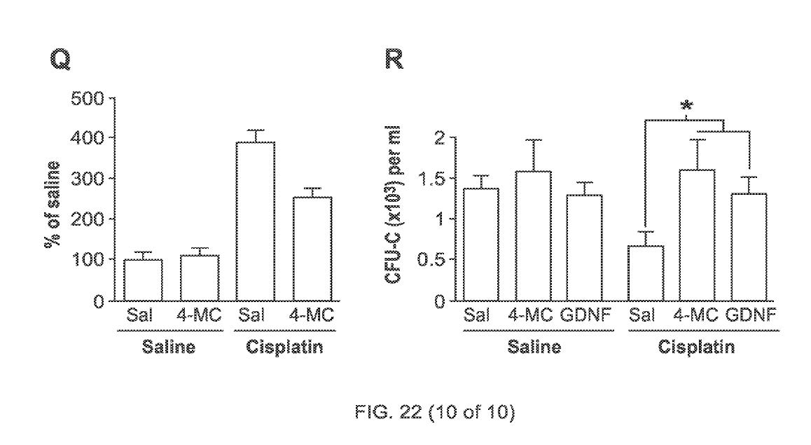

Because bone marrow neuropathy is associated with the deficit in bone marrow regeneration, it followed that interventions that protect neural function would be expected to also restore hematopoietic functions. 4-methylcatechol (4-MC), a drug reported to induce endogenous neural growth factor production and to protect SNS fibers, was administered during 7 cycles of cisplatin chemotherapy (FIGS. 4A and 22A). At four weeks post-transplantation, heated-pad latencies in the cisplatin+saline group were significantly increased, but those of animals treated with cisplatin and 4-MC were comparable to animals that had not received chemotherapy (FIG. 4B). Further, immunofluorescence staining of bone marrow TH⁺ fibers revealed a 2-fold (p<0.05) increase in fiber density in cisplatin+4-MC compared to cisplatin+saline control group (FIG. 4C-D). Moreover, 4-MC accelerated bone marrow regeneration after transplantation, as determined by significant increase in BM cellularity, progenitor counts and Lin⁻Sca1⁺c-kit⁺ft13⁻ cells one month after transplantation (FIG. 4E-G). While a third of cisplatin-treated mice died after transplantation, no death was observed in animals that also received 4-MC (FIG. 4H; p<0.05 Logrank test). These results indicate that protection from cisplatin-induced neuropathy by 4-MC accelerates BM regeneration. To evaluate further whether the effect of 4-MC was specifically due to the recovery of SNS fibers, 4-MC was administered to mice in which the SNS was lesioned with 6OHDA, and BM regeneration was analyzed after 5FU injection (FIG. 7A). Results showed that 4-MC administration also induced TH⁺ fiber recovery (FIG. 7B-C), completely protected from death (FIG. 4I) and also increased hematopoietic recovery (FIG. 4J-L).

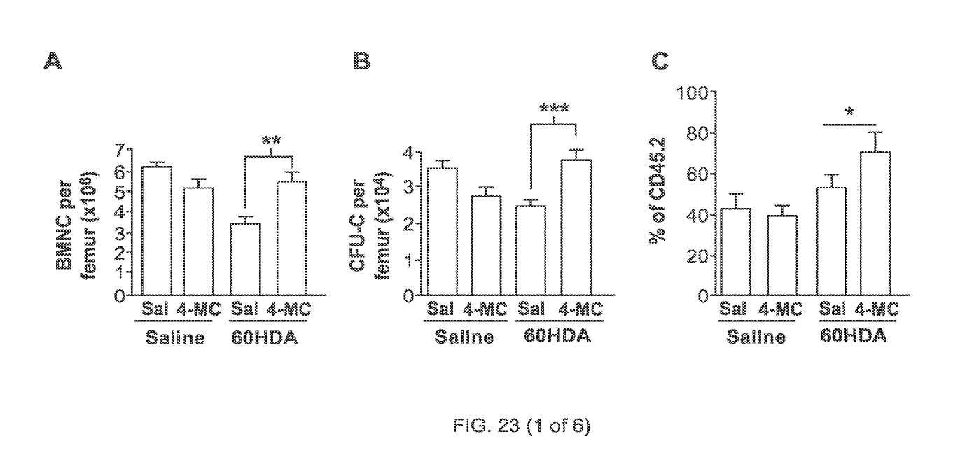

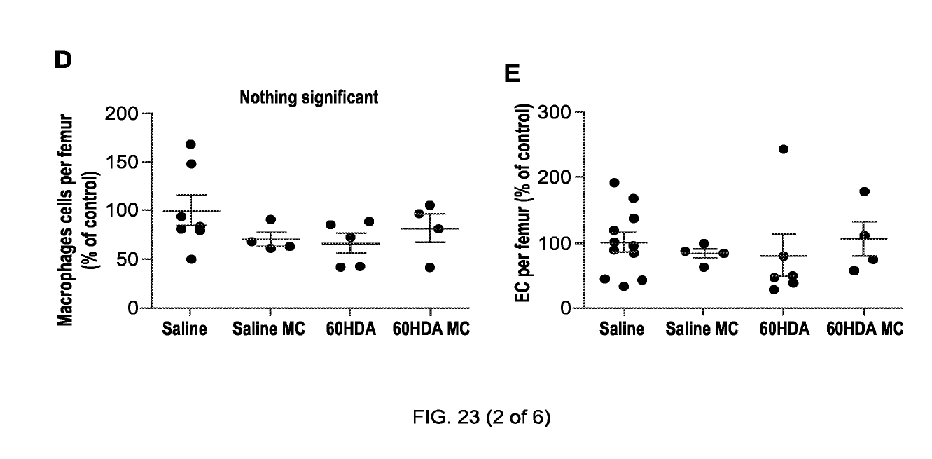

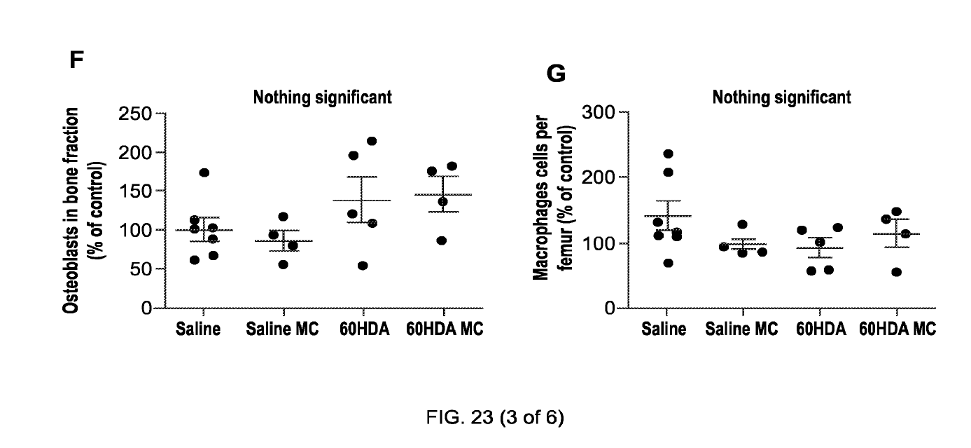

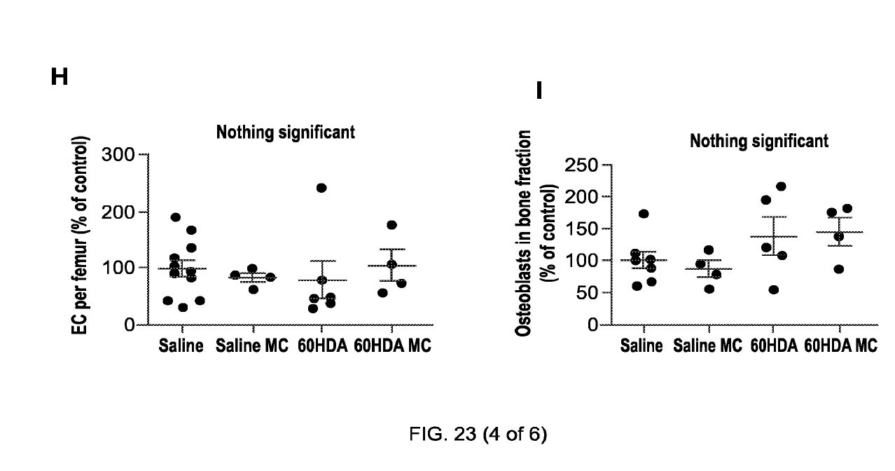

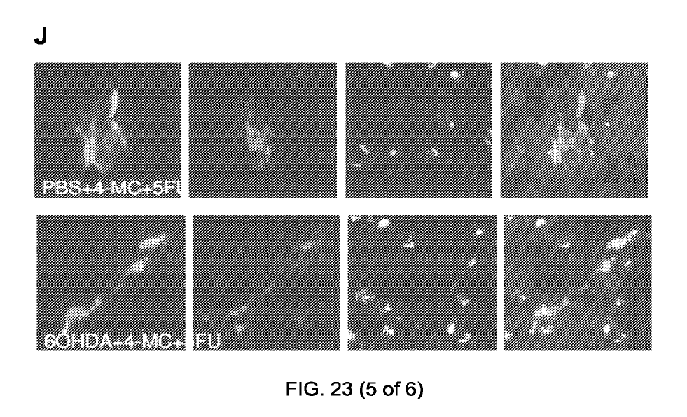

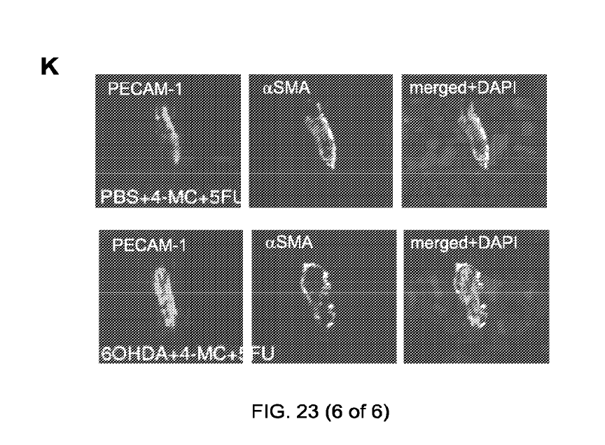

The administrations of 4-MC noted in the preceding paragraph completely abolished 5FU-associated cell death and significantly increased BM (FIGS. 23A-B and FIG. 22H) and LSKF cell recovery (FIG. 22C). 4-MC also induced a significant increase in HSC recovery as measured by competitive repopulation assays (FIG. 23C). In 60HDA-sympathectomized mice, 4-MC treatment resulted in a significant recovery in the number of BM SNS fibers (FIG. 22D-E). In addition, 4-MC abolished the expansion induced by SNS injury in Nestin⁺ cells and osteoblasts prior to 5Fu injection (FIG. 22F; FIG. 23F) and restored normal Nestin⁺ cell number 12 days after 5FU (FIG. 19F) administration. 4-MC treatment did not affect other niche cells, including endothelial cells, BM macrophages, and perivascular α-SMA cells before or after 5FU injection (FIG. 22H and FIG. 23E-K). Since 4-MC acts by increasing NGF, which acts through TrkA receptors, and since the TrkA receptor is expressed by BM cells and can enhance proliferation, an experiment was designed to assess whether increased BM recovery was due to a 4-MC effect on SNS nerves. Tα1-Cre:TrkA^{Neo/Neo} mice, in which TrkA receptor expression is restricted to the nervous system, were used. Sympathectomized Tα1-Cre:TrkA^{Neo/Neo} mice treated with 4-MC showed recovery comparable to that of wild-type mice after 5FU injection (FIG. 221-K). This result showed that 4-MC maintains hematopoietic function by specifically protecting SNS fibers in the BM.

An investigation was also undertaken to determine whether 4-MC could increase BM recovery in cisplatin-treated mice. 4-MC was injected daily for the seven weeks of cisplatin treatment and, after a 4-week recovery period, BMT was performed. Treatment with 4-MC completely abolished transplant-associated death (FIG. 22L) and accelerated bone marrow regeneration after transplantation, as determined by significant increase in BM cellularity, progenitor counts and LSKF cells one month after transplantation (FIG. 22M-O).

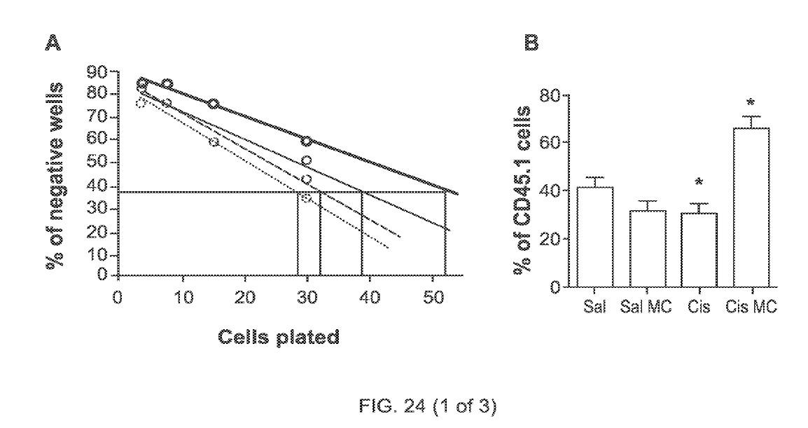

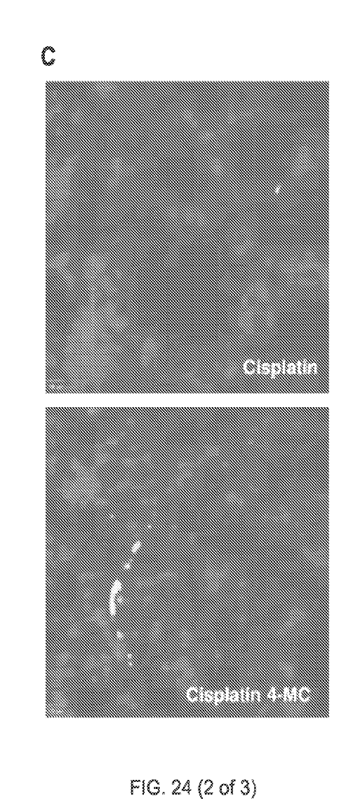

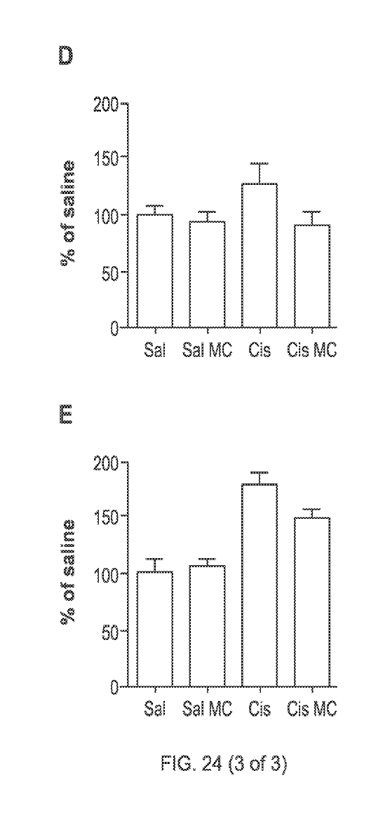

4-MC treatment also resulted in higher frequency and absolute numbers of HSC, as confirmed by LTC-IC (FIG. 24A) and competitive repopulation assays (FIG. 24B). These data correlated with increased SNS innervations, as immunofluorescence stainings of bone marrow TH⁺ fibers revealed a 2-fold (p<0.05) increase in fiber density in cisplatin+4-MC compared to cisplatin+saline control group (FIG. 22P and FIG. 24C). These results indicate that protection from cisplatin-induced neuropathy by 4-MC accelerated BM regeneration. In addition, 4-MC treatment ameliorated the expansion in Nestin⁺ cells (FIG. 22Q) and osteoblasts (FIG. 24E) observed in cisplatin-treated mice before BMT without affecting endothelial cells, BM macrophages, or perivascular α-SMA⁺ cells (FIG. 24D-E).

In addition, a study administering 4-MC or GDNF-Fc to cisplatin-treated mice demonstrated that the anti-neuropathic agents provided sensory neuroprotection. See FIG. 13 and the brief description thereof.

Thus, 4-MC protects SNS fibers in bone marrow and improves hematopoietic regeneration.

### Example 8

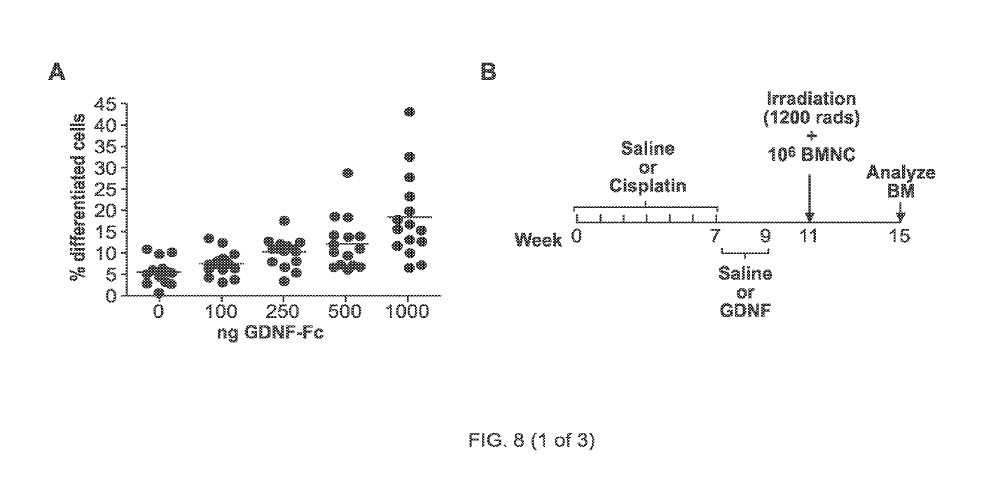

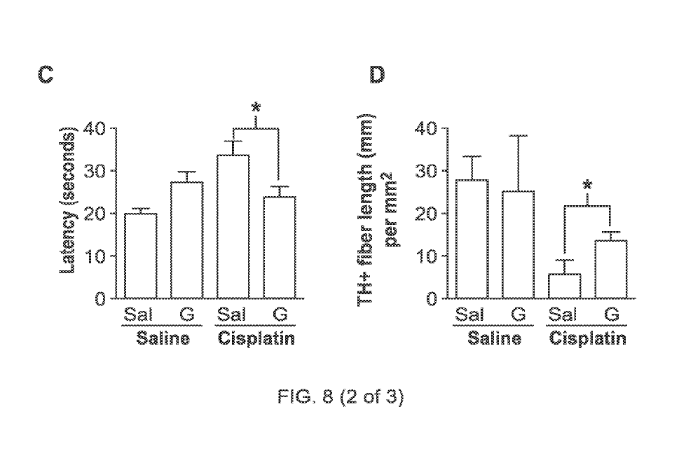

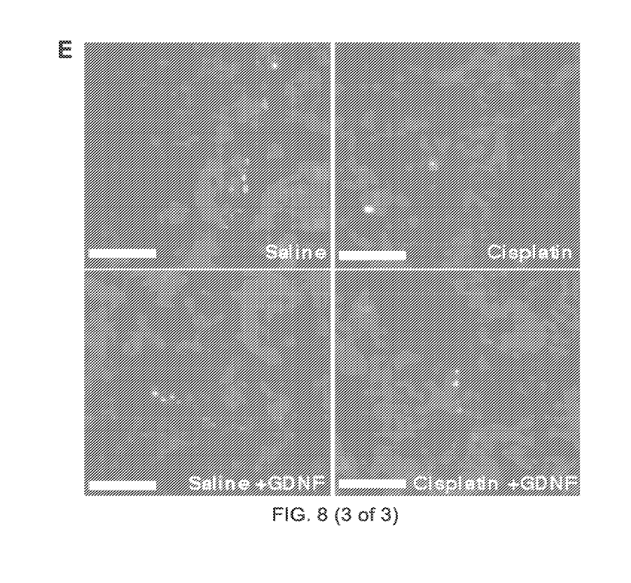

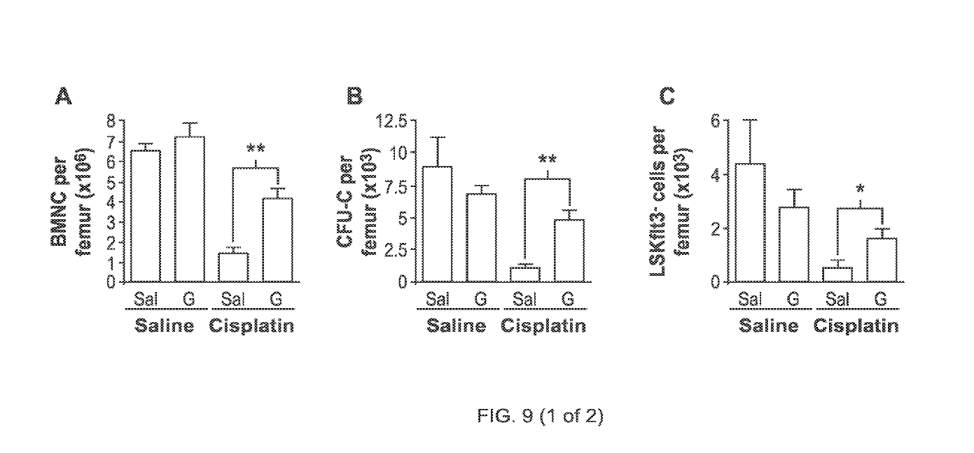

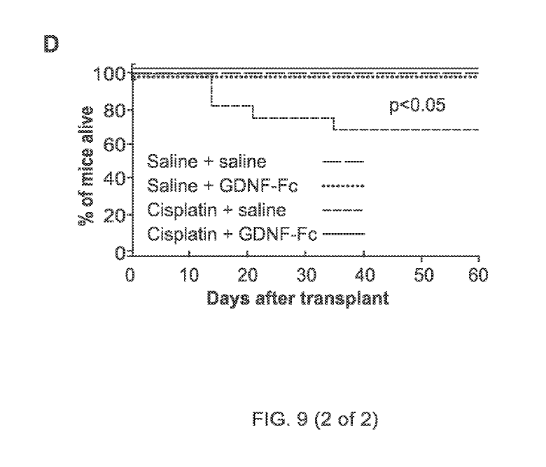

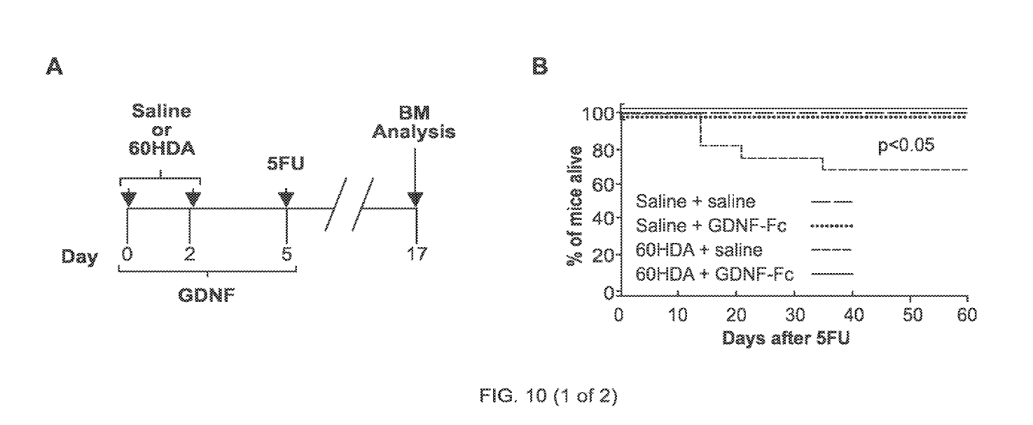

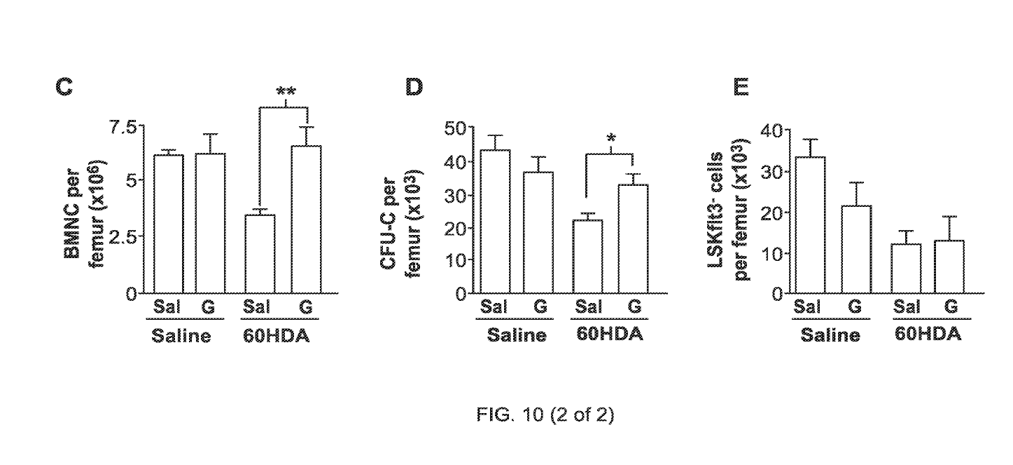

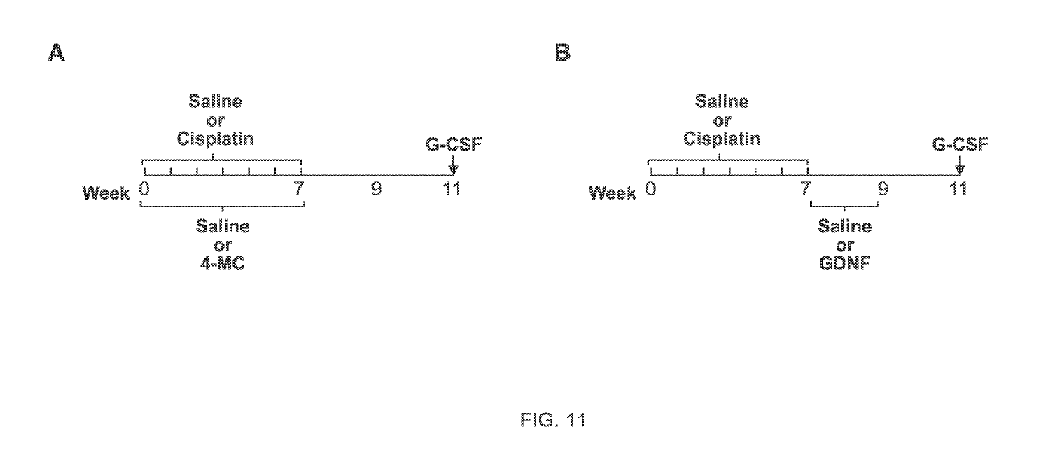

To evaluate further the effect of neuroprotection in a clinically relevant setting, an experiment was conducted to determine whether 4-MC or GDNF-Fc treatment could restore G-CSF-induced mobilization in cisplatin-treated mice. GDNF-Fc is a chimeric molecule engineered by fusion of the C-terminal end of the murine glial cell-derived neurotrophic factor gene (Gdnf), which was reported to rescue preganglionic sympathetic neurons after adrenomedullectomy, with the human IgG1 Fc region. Purified GDNF-Fc was able to induce neural differentiation of PC12ES cells, thus demonstrating its activity in vitro (FIG. 8A). Treatment of mice with daily subcutaneous injections of GDNF-Fc (FIG. 8B) reduced cisplatin-induced sensory neuropathy (FIG. 8C) and improved regeneration of BM TH⁺ fibers compared to mice treated with cisplatin alone (FIG. 8D-E). GDNF-Fc treatment also restored normal hematopoietic recovery after transplantation, as measured by higher bone marrow cellularity, progenitor and HSC counts (FIGS. 9A-C), and improved survival (FIG. 9D). To test the specificity of GDNF-Fc neural regeneration, mice were treated with 60HDA and GDNF-Fc and BM regeneration was analyzed after 5FU injection (FIG. 10A). GDNF-Fc treatment led to a significant improvement in overall survival (FIG. 10B) and hematopoietic recovery (FIG. 10C-E). These data establish that GDNF-Fc acts specifically on SNS fibers to improve hematopoietic regeneration.

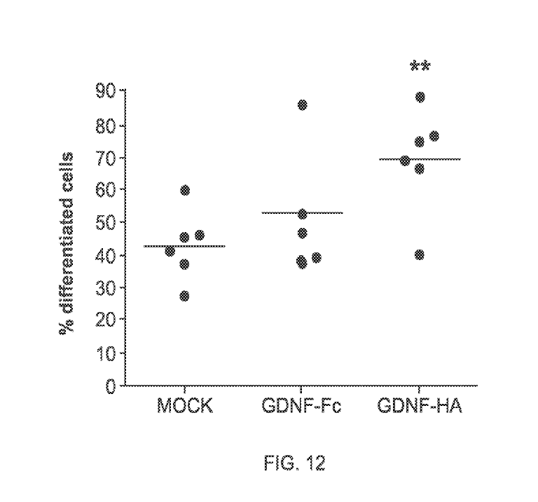

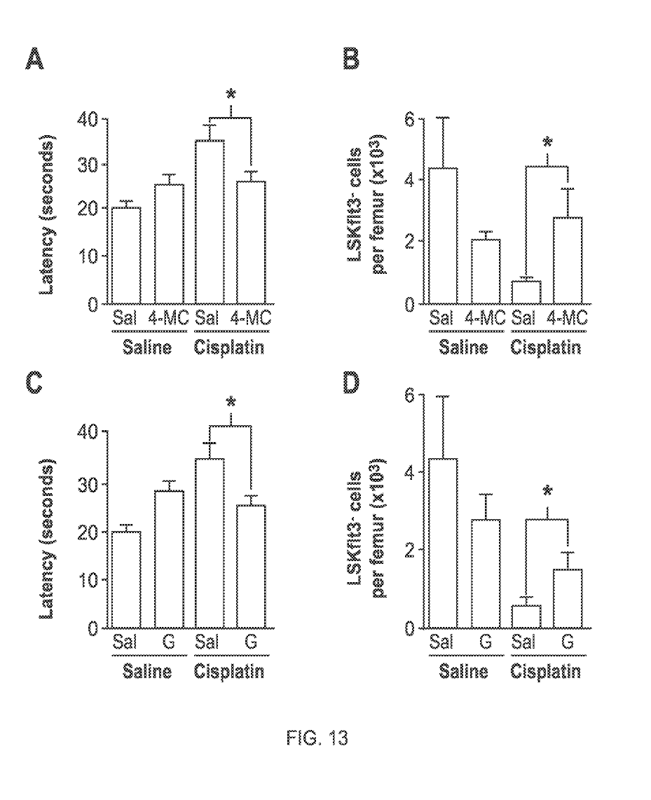

GDNF also was fused to hemagluttinin (GDNF-HA) with similar effect. See FIG. 12. The results of exposing cells to GDNF-HA was an increase in the percent differentiation of exposed cells, establishing that fused GDNF retained biological activity when fused to HA as well as when fused to Fc. Accordingly, it is expected that fusion of anti-neurotrophic agents to fusion partners such as targeting moieties, Fc or HA will yield agents that retain the anti-neuropathic activity and add an activity/ies such as (1) the capacity for targeting specific molecules (e.g., proteins), cells, tissues or organs, (2) an extended in vivo half-life through increased molecular stability and/or decreased clearance rate, and the like. Additionally, it is expected that cytotoxic anti-neuropathic agents may exhibit reduced cytotoxicity when the anti-neuropathic agent is fused to a fusion partner such as a targeting moiety, Fc or HA.

Finally, it is worth noting that, following G-CSF administration to mice, the number of mobilized progenitors in blood was markedly reduced in the group treated with cisplatin (FIG. 22R). In contrast, G-CSF-induced mobilization was completely normalized after neuroprotection with 4-MC (FIG. 22R) or GDNF-Fc (FIG. 22R). These results further confirm the detrimental effects of bone marrow neuropathy in the response to hematopoietic stress. In addition, the data establishes that neuroprotective agents are useful in restoring mobilized progenitor cells following application of a stress such as radio- or chemo-therapy to treat cancer.

### Example 9

To evaluate further the effect of neuroprotection in a clinically relevant setting, 4-MC or GDNF-Fc treatments were analyzed for the ability to restore G-CSF-induced mobilization in cisplatin-treated mice (FIG. 11A-B). Following G-CSF administration, the number of mobilized progenitors in blood was markedly reduced in the group treated with cisplatin (FIG. 4M-N). In contrast; G-CSF-induced mobilization was completely normalized after neuroprotection with 4-MC (FIG. 4M) or GDNF-Fc (FIG. 4N). Thus, these results further confirm the detrimental effects of bone marrow neuropathy in the response to hematopoietic stress and establish that anti-neuropathic agents are effective in recovering from hematopoietic insult.

### Example 10

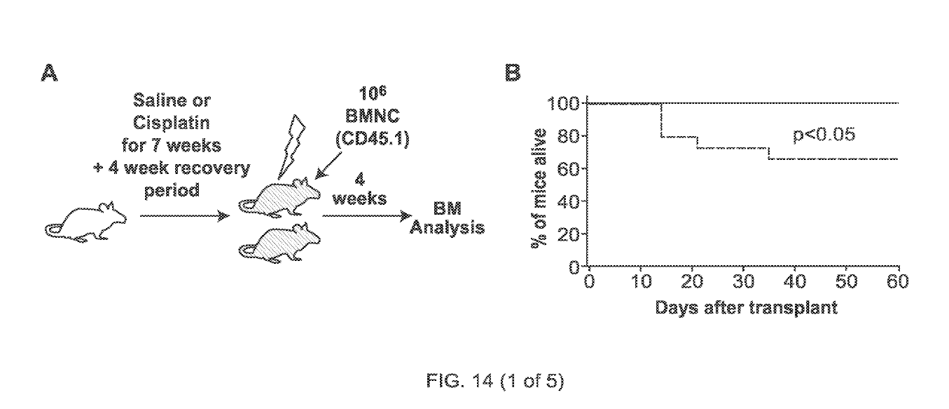

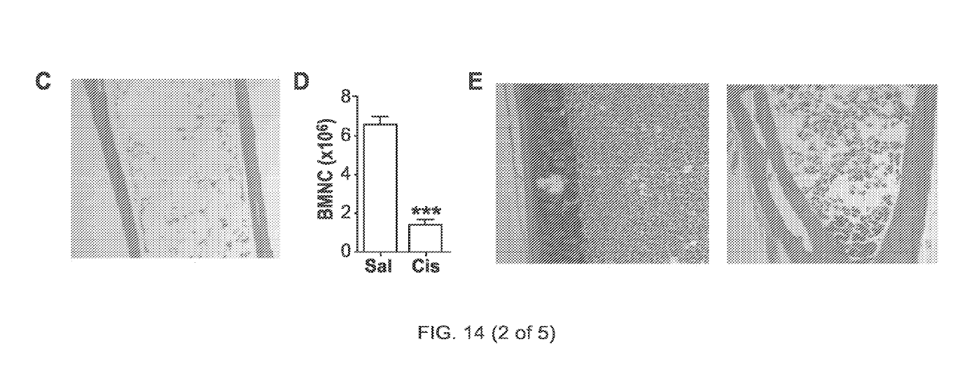

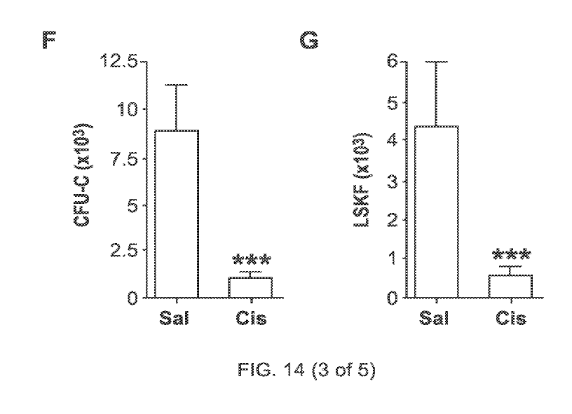

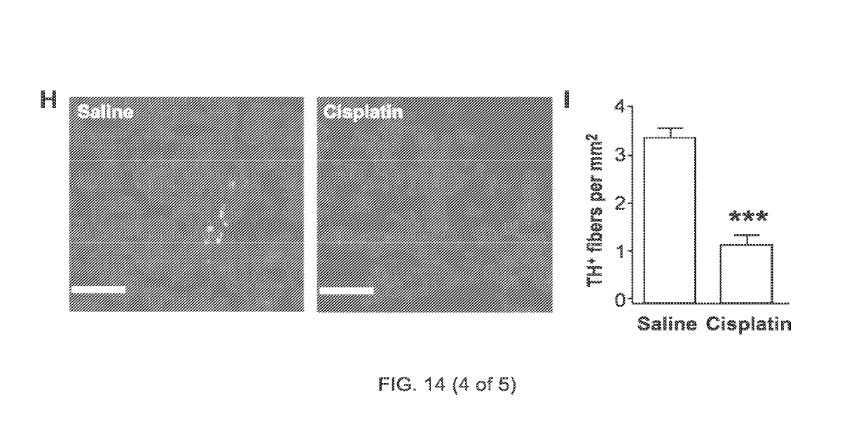

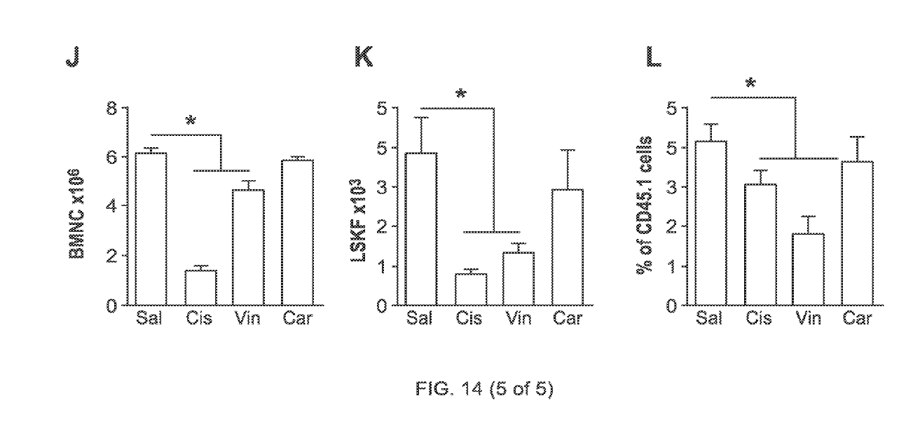

The investigation disclosed in Example 2 was extended using the mouse model of sensory neuropathy induced by cisplatin treatment. As in the study of Example 2, mice were treated with seven weekly injections of cisplatin. One month later, hematopoiesis had completely recovered, as measurements of bone marrow cell, progenitor cell (CFU-C) and Lin⁻Sca1⁺c-kit⁺ cell counts showed that hematopoiesis had completely recovered (FIG. 5A-D). When cisplatin- or vehicle-treated mice were lethally irradiated and transplanted with fresh bone marrow nucleated cells (BMNC) from healthy donors (FIG. 14A), long-term survival in the cisplatin group was significantly reduced (by 33%; FIG. 14B). Increased lethality was due to reduced hematopoietic activity as shown by BM aplasia (FIG. 14C) and severe anemia in moribund mice (Table 2). Four weeks after bone marrow transplantation (BMT), the bone marrow of surviving cisplatin-treated mice was still severely aplastic (FIGS. 14D-E), and showed dramatic reductions in the number of progenitors (FIG. 14F) and Lin⁻Sca1⁺c-kit⁺flt3⁻ (LSKF) cells (FIG. 14G). These data indicate that cisplatin treatment alters the host bone marrow microenvironment, impairing hematopoietic recovery after transplantation of healthy hematopoietic stem and progenitor cells. Cisplatin-induced neuropathy has been reported to affect largely sensory nerves. To assess whether cisplatin also caused a sympathetic neuropathy in the bone marrow, bone marrow SNS fibers were stained with an antibody against the catecholaminergic enzyme tyrosine hydroxylase (TH). Cisplatin treatment reduced the density of TH⁺ fibers by 65% compared with vehicle control (FIG. 14H-I).

TABLE 2
Table 2. Complete blood counts (CBC) of monbund cisplatin-treated mice
at the indicated time points after transplantation of 10⁶ BMNC

| Days after | WBC | RBC | Hgb | HCT | MCV | MCH | MCHC | CHCM | CH | RDW | HDW | PLT | MPV |
|---|---|---|---|---|---|---|---|---|---|---|---|---|---|
| BMT | ×10 [symbol] ml | ×10 [symbol] ml | (g dl) | [symbol] | 1L | pg | g dL | g dL | pg | [symbol] | g dL | ×10 [symbol] ml | 1L |
| 9 | 0.1 | 5.6 | 7 | 20 | 36.3 | 11.9 | 32.8 | 32.1 | 11.6 | 14.8 | 2.32 | 260 | 27.9 |
| 9 | 0.6 | 6.5 | 8 | 27 | 41.1 | 12.3 | 28.9 | 30.8 | 12.6 | 19.8 | 2.38 | 140 | 16.5 |
| 9 | 0.3 | 7.6 | 8 | 30 | 39.1 | 11.1 | 28.3 | 30.5 | 11.9 | 18.1 | 2.19 | 300 | 9.1 |
| 10 | 0.3 | 3 | 3 | 16 | 46.2 | 10 | 21.7 | 31.3 | 14.3 | 26.1 | 3.25 | 40 | 9.6 |
| 12 | 0.8 | 3.2 | 4 | 18 | 56.1 | 11.7 | 20.9 | 27.3 | 15.1 | 30.7 | 3.57 | 30 | 10.1 |
| 12 | 0.3 | 3.8 | 0 | 18 | 46.3 | 0 | 0 | 28.2 | 12.8 | 21.8 | 4.45 | 90 | 20.5 |
| 13 | 0.3 | 4.4 | 4 | 18 | 40.7 | 8.7 | 21.4 | 29.4 | 11.8 | 24.9 | 3 | 240 | 9.9 |

[symbol]

indicates data missing or illegible when filed

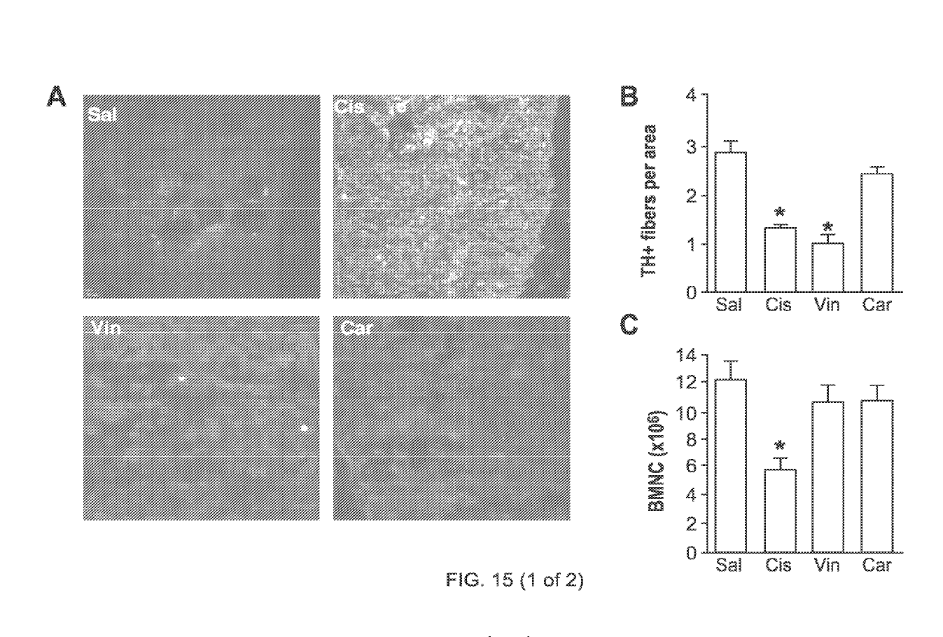

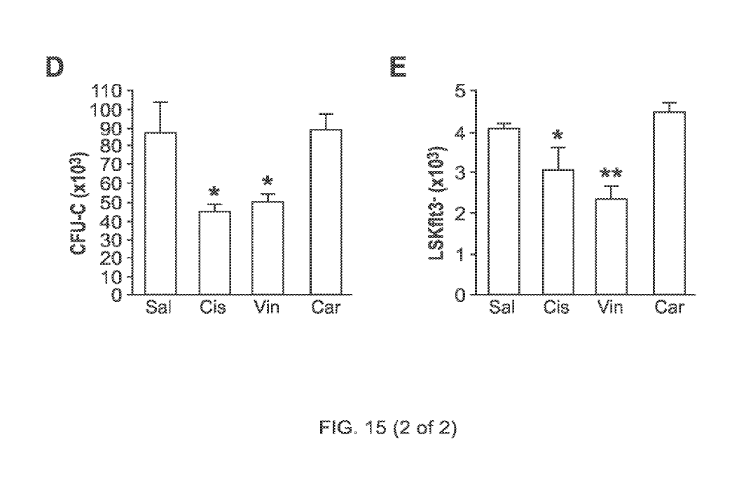

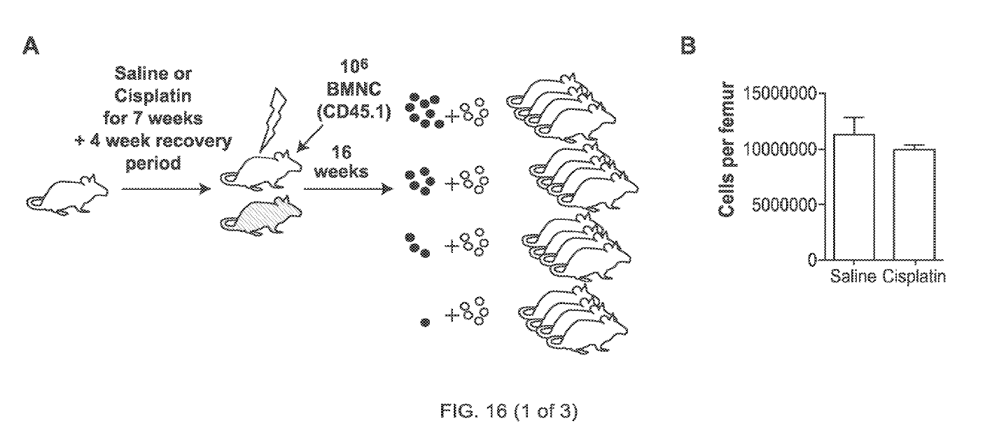

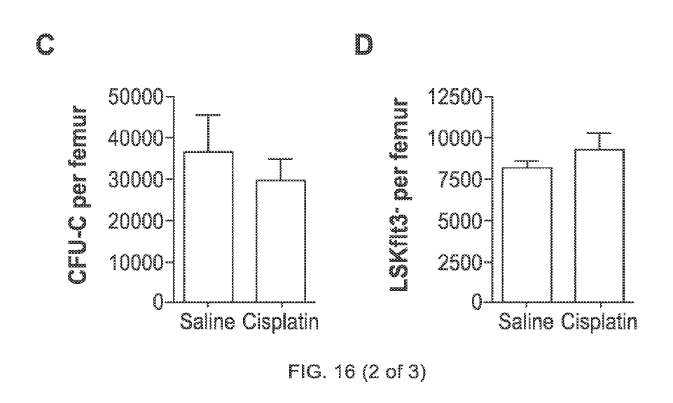

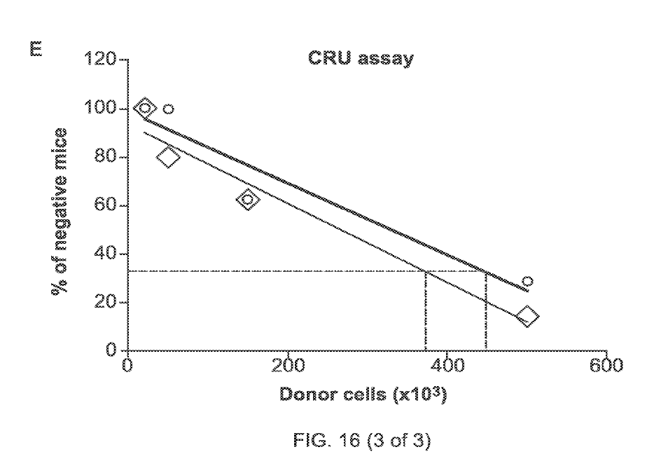

To further investigate whether neurotoxic chemotherapy drugs impaired BM recovery, mice treated with cisplatin, vincristine (which also induces sympathetic neuropathy) and carboplatin (a chemotherapy agent similar to cisplatin but with much reduced neurotoxicity) were compared. In line with expectations based on the disclosures herein, vincristine-, but not carboplatin-, treated mice showed impaired total BM (FIG. 14J) and LSKF cell recovery 4 weeks after transplantation (FIG. 14K). The reduced HSC content in cisplatin- and vincristine-treated mice was confirmed by competitive repopulation assays (FIG. 14L), and this correlated with the degree of SNS injury. This injury is long-lasting and can still be detected 3 months after transplantation (S2A-B), at a time when the BM of cisplatin- and vincristine-treated mice has not yet completely recovered (FIG. 15C-E). Four months after transplantation, no differences were detected in BM cellularity, CFU-C, LSKF cells, or HSC, as measured in competitive reconstitution assays between the BM of control or cisplatin-treated mice (FIG. 16A-E), indicating that cisplatin delays (but does not permanently impair) BM recovery.

The disclosed subject matter has been described with reference to various specific and preferred embodiments and techniques. It should be understood, however, that many variations and modifications may be made while remaining within the spirit and scope of the disclosed subject matter.

1. A method of promoting hematopoietic regeneration in a subject comprising administering an effective amount of a sympathetic nervous system neuroprotective agent.

2. A method of reducing a loss of hematopoietic regeneration capacity in a subject comprising administering an effective amount of a sympathetic nervous system neuroprotective agent.

3. The method according to claim 1 wherein the neuroprotective agent is selected from the group consisting of 4-methylcatechol (4-MC), Glial cell-Derived Neurotrophic Factor, Glial cell-Derived Neurotrophic Factor fusion protein, interleukin-6, insulin growth factor, neural growth factor, vitamin E, glutathione leukemia inhibitory factor, acetylcysteine, acetyl-L-carnitine, amifostine, glutathione, oxcarbazepine, E2072, 2-(Phosphonomethyl) pentanedioic acid, 2-(3-mercaptopropyl)pentanedioic acid, Trypanosoma cruzi trans-sialidase/parasite-derived neurotrophic factor, Brain-Derived Neurotrophic Factor, Transforming Growth Factor-β, cardiotrophin-1, Insulin-like Growth Factor-1, basic Fibroblast Growth Factor, Vascular Endothelial Growth Factor, Hepatocyte Growth Factor Neurotrophin 3, Neurotrophin 4/5, platelet-rich plasma, pifithrin, Z-1-117, 2-imino-2,3,4,5,6,7-hexahydrobenzothiazole derivatives, 2-imino-2,3,4,5,6,7-hexahydrobenzoxazole derivatives, Gambogic amide, amitriptyline, 7,8-dihydroxyflavone, neurturin, artemin, and persephinm.

4. The method according to claim 3 wherein the neuroprotective agent is selected from the group consisting of Glial Cell-Derived Neurotrophic Factor, a Glial Cell-Derived Neurotrophic Factor fusion protein, 4-methylcatechol, interleukin-6, insulin growth factor, neural growth factor, vitamin E, glutathione and leukemia inhibitory factor.

5. The method according to claim 1 wherein the neuroprotective agent is selected from the group consisting of an inhibitor of a glutamate carboxypeptidase, a eukaryotic growth factor, an inhibitor of p53, an agonist of a Trk receptor, an agonist of an RET receptor, and a Glial-Derived Neurotrophic Factor family member.

6. The method according to claim 1 wherein the subject exhibits a stress to hematopoiesis.

7. The method according to claim 1 wherein the subject has received cancer treatment in the form of chemotherapy or radiotherapy.

8. The method according to claim 1 wherein the subject exhibits diabetic neuropathy.

9. The method according to claim 1 wherein the subject is a human.

10. The method according to claim 1 wherein the agent is targeted to a site of hematopoiesis.

11. The method according to claim 1 wherein the agent does not directly contact brain tissue.

12. The method according to claim 1 wherein the agent is unable to restore detectable motor nerve function.

13. The method according to claim 8 wherein the agent is targeted to bone marrow.

14. The method according to claim 1 wherein the agent is administered in a targeting vehicle.

15. The method according to claim 14 wherein the targeting vehicle is selected from the group consisting of a thixotropic gel, a liposome comprising a targeting moiety, an inclusion complex, a micelle and a fused targeting peptide.

16. The method according to claim 14 wherein the agent is contained in a liquid solution, a suspension, an emulsion, a gel, a tablet, a pill, a capsule, a powder, a suppository, a liposome, a microparticle and a microcapsule.

17. The method according to claim 16 wherein the agent is contained in an immediate release formulation, a controlled release formulation, a sustained release formulation, an extended release formulation, a delayed release formulation and a bi-phasic release formulation.

18. The method according to claim 1 wherein the effective amount of the agent is unable to induce regeneration of detectable sympathetic nerve fibers in the bone marrow.

19. A method of improving the mobilization of hematopoietic stem cells in a cancer patient comprising administering a therapeutically effective amount of a sympathetic nervous system neuroprotective agent.


SEQUENCE LISTING (2 sequences)

SEQ ID NO: 1 - DNA, 27 bp, Artificial Sequence
  Synthetic primer
acgctagcaatgggattcgggccactt

SEQ ID NO: 2 - DNA, 30 bp, Artificial Sequence
  Synthetic primer
cgagatctgcgatacatccacaccgtttag